(19) United States
(12) Patent Application Publication
(10) Pub. No.: US 2025/0001050 A1
(43) Pub. Date: Jan. 2, 2025

(54) COMPOSITIONS AND METHODS FOR TREATING BONE INJURIES

(71) Applicant: BOARD OF REGENTS, THE UNIVERSITY OF TEXAS SYSTEM, Austin, TX (US)

(72) Inventor: Vaida Glatt, San Antonio, TX (US)

(21) Appl. No.: 18/688,180

(22) PCT Filed: Aug. 31, 2022
(86) PCT No.: PCT/US2022/042258

§ 371 Date: Feb. 29, 2024

Related U.S. Application Data
(60) Provisional application No. 63/239,161, filed on Aug. 31, 2021.

(51) Int. Cl. A61L 27/54; A61L 27/36; A61L 27/44; A61L 27/56

(52) U.S. Cl. CPC A61L 27/54; A61L 27/3608; A61L 27/3616; A61L 27/365; A61L 27/44; A61L 27/56; A61L 2300/414; A61L 2430/02

(57) ABSTRACT

The disclosure relates to compositions and biomimetic scaffolds and methods of using to treat, improve, and accelerate the healing of large segmental bone defects in a subject. The methods comprise implanting said compositions and biomimetic scaffolds in a subject

### CROSS REFERENCE TO RELATED APPLICATIONS

This application claims the benefit of the filing date of U.S. Provisional Application No. 63/239,161, filed on Aug. 31, 2021. The content of this earlier filed application is hereby incorporated by reference in its entirety.

### BACKGROUND

On average 7.9 million fractures occur annually in the U.S. alone, with approximately 5-10% resulting in delayed unions, non-unions, sub-critical size defects or large bone defects, creating significant therapeutic challenges for surgeons (Zura, et al., JAMA Surg. 2016 Nov. 16; 151). In the general population, fracture severity, anatomic location, patient comorbidities, smoking, and the use of certain medications can contribute substantially towards problems with fracture healing (Zura, et al., JAMA Surg. 2016 Nov. 16; 151). The cost of treatment of non-unions of the tibia, femur, or humerus ranges between $31,500 and $34,400 for each case, a substantial burden on health care costs annually (Kanakaris N K, Giannoudis P V. Injury. 2007; 38 Suppl 2: S77 S84; and Wu, et al Orthopedic Research and Reviews. 2013:5 21-33).

In addition, more than 59,000 military personnel have been wounded in combat related operations while serving in Iraq and Afghanistan, of which 50% were musculoskeletal injuries, including large segmental bone defects (Belmont Jr. P J, McCriskin B J, Sieg R N, Burks R, Schoenfeld A J. Combat wounds in Iraq and Afghanistan from 2005 to 2009. J Trauma Acute Care Surg. 2012; 73: 3-12). It has been estimated that 78% of musculoskeletal injuries sustained in these conflicts were major limb injuries (Stansbury L G, Lalliss S J, Branstetter J G, Bagg M R, Holcomb J B. Amputations in U.S. military personnel in the current conflicts in Afghanistan and Iraq. J Orthop Trauma. 2008; 22: 43-46). Such bone injuries heal poorly, specifically those related to high impact blast injuries due to contaminated bone and soft tissue destruction combined with locally decreased tissue vascularity. Even without such complications, bone defects beyond a critical size have no intrinsic ability to heal. The management of patients with large segmental bone defects is still one of the most challenging clinical problems faced by both military and civilian surgeons (Pollak A N, Ficke J R, Extremity War Injuries III Session Moderators. Extremity war injuries: challenges in definitive reconstruction. J Am Acad Orthop Surg. 2008; 16: 628-34). The importance of improving the ability of clinicians to successfully manage these injuries cannot be overemphasized, as these devastating injuries often result in a protracted course of continued medical care that is inevitably costly, with a significant risk of failure and eventual amputation. As a result of the military activity in Iraq and Afghanistan alone (OIF/OEF), more than two thousand soldiers have required at least one amputation (Fischer H. A Guide to U.S. Military Casualty Statistics: Operation Freedom's Sentinel, Operation Inherent Resolve, Operation New Dawn, Operation Iraqi Freedom, and Operation Enduring Freedom. Congr Res Serv. 2015; 7). Furthermore, persistent non-healing bone defects are associated with a delayed return to work or military duties, with a corresponding reduction in the individual's quality of life. Therefore, there is clearly an unsatisfied clinical need, a powerful motivator towards development of more effective treatment strategies to address these potentially catastrophic injuries.

### SUMMARY

Disclosed herein are compositions comprising: an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride; and a bone substitute.

Disclosed herein are compositions comprising: an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride; and a bone substitute.

Disclosed herein are multi-compartment devices comprising a first chamber comprising isolated whole blood and/or one or more growth factors, one or more bone substitutes, or a combination thereof, a second chamber comprising ecarin or calcium chloride; thrombin; or thrombin and calcium chloride.

Disclosed herein are biomimetic scaffolds comprising a scaffold and an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride.

Disclosed herein are biomimetic scaffolds comprising a scaffold and an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride.

Disclosed herein are methods of constructing an implant, the method comprising: a) dimensioning a depot implant in at least one of a shape and a size that facilitates implantation of the depot implant into a bone defect; and b) structuring the depot implant to have a scaffold by introducing: (i) isolated whole blood and sodium citrate; or platelet rich plasma, plasma, or plasma with red blood cells; (ii) ecarin; oscutarin and calcium chloride; calcium chloride; thrombin; or thrombin and calcium chloride; and (iii) a bone substitute to create the scaffold.

Disclosed herein are methods of constructing a biomimetic scaffold, the methods comprising: a) dimensioning a scaffold in at least one of a shape and a size that facilitates implantation of the scaffold into a bone defect; and b) combining the scaffold in a) with an ex vivo hematoma comprising: (i) isolated whole blood and sodium citrate; or platelet rich plasma, plasma, or plasma with red blood cells; and (ii) ecarin; oscutarin and calcium chloride; calcium chloride; thrombin; or thrombin and calcium chloride to create the biomimetic scaffold.

Other features and advantages of the present compositions and methods are illustrated in the description below, the drawings, and the claims.

### BRIEF DESCRIPTION OF THE DRAWINGS

FIG. 1 shows healing outcomes of 5 mm femoral defects in rats 8 weeks after surgery. ACS—absorbable collagen sponge, BMP2—bone morphogenetic protein 2, MicroCT—microcomputed tomography, WB—whole blood.

FIG. 2 shows the micro-architecture of demineralized bone matrix (DBX mixed with an ex vivo hematoma (BH) at—at a ratio of DBX:BH of 1:3 and 1:6.

FIG. 3 shows an ex vivo hematoma delivered within a 3D printed titanium custom spinal cage.

FIG. 4 shows a 3D patient specific titanium femoral implant (cages/scaffolds) that can be used as disclosed herein.

FIG. 5 shows an example of a two-chamber syringe that can be used to deliver components of an ex vivo hematoma. One chamber can deliver the coagulants, and the other chamber can deliver either whole blood alone or in combination with growth factors and/or bone substitutes.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder or condition. The term “patient” includes human and veterinary subjects. In some aspects of the disclosed methods, the “patient” has been diagnosed with a need for treatment for healing of bone injuries, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a bone injury or a bone fracture.

Current methods of treating large segmental bone defects. Many techniques have been employed for the treatment of skeletal defects, including autologous bone grafts, various bone graft substitutes, Ilizarov methods, arthroplasty with mega-prostheses, biologics, and as a last resort, amputation. However, these existing treatment options characteristically require prolonged treatment involving multiple surgeries, are expensive, have high complication rates, and are associated with a significant risk of treatment failure. For example, autografting remains the treatment of choice when healing large segmental bone defects (Khan S N, Cammisa F P, Sandhu H S, Diwan A D, Girardi F P, Lane J M. J Am Acad Orthop Surg. 2005; 13: 77-86). Autologous bone grating remains the treatment of choice when treating bone defects, however, the supply of sufficient autograft material can be limited, especially in the case of severe traumatic injury or nonunion of fracture in a civilian population and severely injured soldiers, and is associated with significant morbidity at the donor site. In contrast, although allograft bone is available in large quantities, its use raises concerns about disease transmission and the immune response (Khan S N, Cammisa F P, Sandhu H S, Diwan A D, Girardi F P, Lane J M. J Am Acad Orthop Surg. 2005; 13: 77-86), but more importantly, it consists of dead bone that does not resorb or remodel well. It has also been shown to gradually accumulate micro-fractures when loaded, leading to a 5-year failure rate of 30% or greater (Enneking W F, Campanacci D A. J Bone Joint Surg Am. 2001; 83-A: 971-986; and Wheeler D L, Haynie J L, Berrey H, Scarborough M, Enneking W. Biomed Sci Instrum. 2001; 37: 251-256). Additionally, various bone substitutes have also been developed to aid in the therapy of large segmental bone defects, yet these approaches are limited to marginal improvement in outcomes. Inadequate bone formation, poor mechanical and handling properties, a lack of biocompatibility, unpredictable resorption, and associated inflammatory reactions remain major limitations for such materials (McKee M D. J Am Acad Orthop Surg. 2006; 14: S163-7). For this reason, distraction osteogenesis remains the treatment of choice by military surgeons for bone defects that exceed 8 cm. This technique is, however, cumbersome, painful and unreliable, complicated by pin track infections, and can be slow to heal (Pollak A N, Ficke J R, J Am Acad Orthop Surg. 2008; 16: 628-34). Above all, the main complication of the available therapeutic options for large segmental bone defects is their inability to ensure a quick restoration of function, and the ability to reduce the rate of refracture.

Studies into the biology of osteogenesis has led to the discovery of bone morphogenetic proteins (BMPs), which are some of the most potent inducers of osteogenesis (Urist M R. J Bone Miner Res. 1997; 12: 343-6). Two members of the BMP family have been approved for clinical use: recombinant human BMP-2 (rhBMP-2; INFUSE®) and recombinant human BMP-7 (rhBMP-7; OP-1®). Although BMPs have shown preclinical efficacy in animal models, their clinical effectiveness has been disappointing. The modest clinical response to these proteins has been associated with delivery problems. Furthermore, the current practice of using extremely high supra-physiological doses of BMPs, most of which rapidly leaches away from the site of application, has the potential to increase the incidence and severity of ectopic/heterotopic ossification and many other related side effects, such as antibody formation, implant dislodgment, bone resorption and even cancer (Carragee E J, Hurwitz E L, Weiner B K. Spine J. 2011; 11: 471-491). Therefore, a suitable carrier needs to be developed to improve the effectiveness of BMPs by significantly minimizing the dose required, thus limiting their potential side effects and associated treatment costs.

One of the most promising alternatives to autologous bone grafting is the use of recombinant human bone morphogenetic protein 2 (rhBMP-2) delivered on an absorbable collagen sponge (ACS). However, successful bone healing requires a supra-physiological dose of rhBMP-2 due to its burst release and short half-life in vivo, most of which rapidly leaches away as soon as the collagen sponge becomes compressed after insertion into the bone defect area. This results in an increased incidence and severity of ectopic/heterotopic ossification and many other related side effects, such as antibody formation, implant dislodgment, bone resorption, and even cancer. A more suitable carrier is clearly required to treat these complex bone injuries and to improve the effectiveness of BMPs by significantly reducing the dose required, thus mitigating their potentially dangerous side effects.

In recent years, platelet-rich plasma (PRP), blood plasma enriched with a high concentration of platelets, has been trialed across a range of applications, including the treatment of musculoskeletal injuries. PRP is thought to secrete a variety of growth factors and cytokines at supra-physiologic concentrations and generates a cell-supporting matrix in the form of a fibrin clot. Specifically, it has been suggested that the effects of PRP to promote bone healing (Iqbal J, Pepkowitz S H, Klapper E. Curr Osteoporos Rep. 2011; 9: 258-263; and Kurikchy M Q, Al-Rawi N H, Ayoub R S, Mohammed S S. Clin Oral Investig. 2013; 17: 897-904) are due to an increased concentration of factors such as platelet-derived growth factor (PDGF), transforming growth factor beta (TGF-0), bone morphogenetic proteins (BMPs) and insulin-like growth factor 1 (IGF-1) (Soffer E, Ouhayoun J P, Anagnostou F. Oral Surgery, Oral Medicine, Oral Pathology, Oral Radiology, and Endodontics. 2003. pp. 521-528).

Nevertheless, the results from experimental (Simman R, Hoffmann A, Bohinc R J, Peterson W C, Russ A J. Ann Plast Surg. 2008; 61: 337-44) and clinical studies (Marx R E, Carlson E R, Eichstaedt R M, Schimmele S R, Strauss J E, Georgeff K R. Oral Surg Oral Med Oral Pathol Oral Radiol Endod. 1998; 85: 638-46) using PRPs in oral and maxillofacial bone grafting procedures are controversial, and to date there is little evidence to suggest it actually improves or accelerates bone healing, in fact, in the majority of cases there was a decrease in bone formation (Choi B-H, Im C-J, Huh J-Y, Suh J-J, Lee S-H. Int J Oral Maxillofac Surg. 2004; 33: 56-9; and Marden L J, Fan R S P, Pierce G F, Reddi A H, Hollinger J O. J Clin Invest. 1993; 92: 2897-2905). This apparent lack of efficacy also extends to long bones and spine. A recent study in sheep, using an osteotomy of the femoral diaphysis followed by distraction osteogenesis, failed to show that the application of PRP improved new bone formation (Hernandez-Femandez A, Velez R, Soldado F, Saenz-Rios J C, Barber I, Aguirre-Canyadell M. Injury. 2013; 44: 901-7). Likewise, in rodent studies, PRPs have no beneficial effect on bone healing (Pryor M E, Yang J, Polimeni G, Koo K, Hartman M J, Gross H, et al. J Periodontol. 2005; 76: 1287-1292; and Ranly D M, Lohmann C H, Andreacchio D, Boyan B D, Schwartz Z. J Bone Joint Surg Am. 2007; 89: 139-147) or, at best, indicated a low regenerative potential (Sánchez AR, Sheridan P J, Eckert S E, Weaver A L. J Periodontol. 2005; 76: 1637-1644). In studies to encourage bone healing after spinal fusion there were also no benefits in either animals (Li H, Zou X, Xue Q, Egund N, Lind M, Binger C. Eur Spine J. 2004; 13: 354-8) or human subjects (Weiner B K, Walker M. Spine (Phila Pa 1976). 2003; 28: 1968-1970). The factors responsible for the variable outcomes in these studies are not known, but it has been suggested that proteases present in the platelets may degrade the growth factors (Thibault L, Beauséjour A, De Grandmont M J, Lemieux R, Leblanc J F. Transfusion. 2006; 46: 1292-1299) thereby changing the composition of the PRP and reducing its experimental and clinical effectiveness. Furthermore, PRP activation requires thrombin that leads to a burst release of growth factors, and a reduction of total growth factor concentrations. Bovine thrombin interferes with human coagulation proteins by stimulating antibodies against thrombin, which is thought to affect the healing process (Oryan A, Alidadi S, Moshiri Expert Opin Biol Ther. 2016; 16: 213-32). This may be also related to the inability of PRP to form a clot with suitable structural properties.

Based on the poor performance of PRPs, a “second generation” of platelet concentrate was developed by the French maxillofacial surgeon Joseph Choukroun called platelet-rich fibrin (PRF) (Choukroun J, Diss A, Simonpieri A, Girard M-O, Schoeffler C, Dohan S L, et al. Oral Surg Oral Med Oral Pathol Oral Radiol Endod. 2006; 101: 299-303). It was described as a more natural fibrin matrix containing the constituents of blood that are favorable to healing and immunity, with specific advantages over PRPs. For example, the clotting process takes place by a slower natural polymerization compared to the fast polymerization resulting when thrombin is added to PRPs. Most importantly, PRFs do not require anticoagulants or thrombin additives (Dohan D M, Choukroun J, Diss A, Dohan S L, Dohan A J J, Mouhyi J, et al. Oral Surgery, Oral Med Oral Pathol Oral Radiol Endodontology. 2006; 101). In vitro studies have also shown that PRFs have a more sustained release of growth factors such as PDGF, TGF-β, and BMPs, lasting up to 28 days (He L, Lin Y, Hu X, Zhang Y, Wu H. Oral Surgery, Oral Med Oral Pathol Oral Radiol Endodontology. 2009; 108: 707-713). This release profile is significantly different to PRPs, which are characterized by a burst release of cytokines and growth factors within one day. Despite that, PRFs have not shown any significant improvements when applied in oral and maxillofacial surgery (Choukroun J, Diss A, Simonpieri A, Girard M-O, Schoeffler C, Dohan S L, et al. Oral Surg Oral Med Oral Pathol Oral Radiol Endod. 2006; 101: 299-303; and Busenlechner D, Huber C D, Vasak C, Dobsak A, Gruber R, Watzek G. Clin Oral Implants Res. 2009; 20: 1078-1083). Again, this could be related to an insufficient amount of platelets delivered, along with unsuitable structural properties, similar to PRPs.

The influence of hematoma formation on bone fracture healing. Fracture hematoma formation occurs within minutes of a bone injury and includes a cascade of biological events that engage activated coagulation factors from the hematological system as well as a number of molecular factors derived from the periosteum, bone marrow, and surrounding soft tissues, such as pro-inflammatory cytokines and growth factors, including osteogenic and angiogenic factors (Lai B F L, Zou Y, Brooks D E, Kizhakkedathu J N. Biomaterials. Elsevier Ltd; 2010; 31: 5749-5758). At a fracture site, blood vessels contract to prevent sustained blood loss, followed by a coagulation cascade that leads to the formation of a hematoma, or blood clot, situated between the broken fragments (Schindeler A, McDonald M M, Bokko P, Little D G. Semin Cell Dev Biol. 2008; 19: 459-66). In the clotting process, there are two main pathways: intrinsic and extrinsic, in which the common pathway is the conversion of coagulation factor X into Xa. Thrombin, the final protease in the coagulation cascade, is a typical serine protease of the chymotrypsin family and has both pro- and anti-coagulant functions (Huntington J A. Thrombin plasticity. Biochim Biophys Acta-Proteins Proteomics. Elsevier B. V.; 2012; 1824: 246-252). During normal blood coagulation, a coagulation cascade activates the zymogen prothrombin by converting it into the serine protease thrombin. Thrombin then converts the soluble fibrinogen into insoluble fibrin fibers. Finally, these fibrin fibers contribute to the formation of a mature web-like fibrin clot with the assistance of coagulation factor XIII (Chernysh I N, Nagaswami C, Purohit P K, Weisel J W. Sci Rep. 2012; 2: 879). Thus, the concentration gradients of fibrinogen, thrombin, and coagulation factor XIII play an important role in modulating fibrin clot conformation (Wolberg A S, Campbell R A. Transfus Apher Sci. 2008; 38: 15-23). The structural parameters in fibrin clots can be characterized by the fiber diameter, density, the number of branch points, distances between branch points, and dimension of the pores (Weisel J W, Litvinov R I. Blood. 2013; 121: 1712-1719). The rise in diameter of fibrin fiber is inversely proportional to the density of the fibrin fiber and directly proportional to pore size (Eichhom S J, Sampson W W. J R Soc Interface. 2005; 2: 309-318; and Kaur S, Sundarrajan S, Rana D, Matsuura T, Ramakrishna S. J Memb Sci. 2012; 392-393: 101-111). In addition, the diameter and density of fibers has an impact on the porosity and surface area of fibrin clots (Pham Q P, Sharma U, Antonios G. Mikos. Biomacromolecules. 2006; 7: 2796-2805) and is responsible for the biological functions of stem cells, such as adhesion, proliferation, and differentiation (Badami A S, Kreke M R, Thompson M S, Riffle J S, Goldstein A S. Biomaterials. 2006; 27: 596-606). For example, low thrombin concentrations (<1 nM) generate a porous network of thick fibrin fibers highly susceptible to fibrinolysis, whereas high concentrations of thrombin result in thin fibers that form a poorly permeable fibrin network relatively resistant to fibrinolysis (Gabriel D A, Muga K, Boothroyd E M. J Biol Chem. 1992; 267: 24259-63). Moreover, although an individual thick fiber has a higher mechanical strength (stiffness), a fibrin clot composed of thick fibers often has lower mechanical strength as a result of a reduced number of fibers (Carlisle C R, Coulais C, Guthold M. Acta Biomater. 2010; 6: 2997-3003; and Liu W, Thomopoulos S, Xia Y. Adv Healthc Mater. 2012; 1: 10-25).

Combining a scaffold with the disclosed ex vivo hematomas can be used to successfully and efficiently enhance bone regeneration in large segmental defects, which do not heal spontaneously on their own. In some aspects, said combination can be used with about 33× less rhBMP-2 than the standard dose (11 μg) currently used. As described herein, using an in vivo 5 mm femoral defect rat model, rhBMP2 (e.g., 0.33 μg) delivered via an ex vivo hematoma” was much more efficient than when delivered on an absorbable collagen sponge. The large bone defects healed consistently with a markedly lower dose of rhBMP-2, and the quality of the healed bone was also superior in the ex-vivo hematoma group, based on the callus size and the bone morphometric parameters at the end of 8-weeks compared to rhBMP-2 delivered on an absorbable collagen sponge and without being a part of the ex-vivo hematoma. Disclosed herein are biomimetic scaffolds comprising a scaffold and an ex vivo hematoma. Disclosed herein are biomimetic scaffolds comprising a scaffold and an ex vivo hematoma that can be designed and made to mimic a normal fracture hematoma but that more efficiently deliver a very low dose of rhBMP-2 along with a specific concentration of calcium and thrombin or ecarin. Other osteogenic proteins can also be included in the ex vivo hematomas described herein. Designing and using a biomimetic scaffold comprising a scaffold and an ex vivo hematoma can be made to mimic a hematoma that forms upon a fracture, at least because the hematoma formed at the time of injury serves as a scaffold that activates a cascade of biological events. To initiate fracture healing, this cascade can engage in activating coagulation factors from the hematological system, and also a number of molecular factors derived from the periosteum, bone marrow, and surrounding soft tissues, such as pro-inflammatory cytokines and growth factors, as well as osteogenic and angiogenic factors. The hematoma that forms in the biomimetic scaffold at the fracture site can significantly influence the way broken bones heal, while removal of this hematoma would delay fracture healing.

Disclosed herein are methods to improve the structural and biological properties of an induced blood clot in order to enhance the healing of large segmental bone defects. For example, to considerably improve and accelerate the healing of large segmental bone defects in soldiers and civilians, described herein are biomimetic scaffolds comprising scaffolds an ex vivo hematoma created by constructing a fibrin clot that mimics the structural properties of innate fracture hematoma.

Prothrombotic snake venoms. A number of snake venom toxins contain proteolytic enzymes that affect hemostasis by the action of coagulant thrombin-like enzymes and prothrombin activating toxins. The conversion of factor X to (activated) Xa is the common junction between the extrinsic and intrinsic coagulation pathways. Snake venom enzymes have evolved to exploit the various phases of the coagulation cascade. These venoms have evolved to exploit the vulnerable interaction that exists between platelets, endothelial cells and plasma proteins with venom proteins that have an effect on phases of vertebrate hemostasis (Meier J, Stocker K. Crit Rev Toxicol. 1991; 21: 171-182). Depending on the type of snake, they each have specific pro-coagulating factors. For example, the pro-coagulating factor oscutarin found in the venom of the coastal taipan (Oxyuranus scutellatus) is structurally and functionally similar to mammalian coagulation factor X. Oscutarin is a serine protease that belongs to the group C prothrombin activator venoms which, unlike mammalian factor X, does not require the non-enzymatic factor V, since they contain their own factor Va-like molecule (St. Pierre L, Masci P P, Filippovich I, Sorokina N, Marsh N, Miller D J, et al. Comparative analysis of prothrombin activators from the venom of Australian elapids. Mol Biol Evol. 2005; 22: 1853-1864). Similarly, ecarin, found in the venom of the Saw-scaled viper (Echis carinatus) is a metalloproteinase that acts without the involvement of co-factors such as calcium chloride (CaCl₂)) and phospholipids. The lack of co-factor requirement allows ecarin to convert both carboxylated and decarboxylated prothrombin into meizothrombin (Hutton R. Blood Rev. 1993; 7: 176-189). Meizothrombin is an intermediate product of thrombin generation during whole blood clotting and is reported to have limited enzymatic activity in the conversion of fibrinogen to fibrin (Bovill E G, Tracy R P, Hayes T E, Jenny R J, Bhushan F H, Mann K G. Arterioscler Thromb Vasc Biol. 1995; 15: 754-758; and Krishnaswamy S, Mann K G, Nesheim M E. J Biol Chem. 1986; 261: 8977-8984). On the other hand, a serine protease RVV-V isolated from Russell's viper (Dabioa russelli) venom specifically activates factor V in a calcium-independent manner. Another well-characterized protease RVV-X, also isolated from Russell's viper venom, is a potent and specific activator of factor X. In contrast to RVV-V, the RVV-X is a metalloproteinase that requires Ca²⁺ but not phospholipids as a co-factor (Takeya H, Nishida S, Miyata T, Kawada S I, Saisaka Y, Morita T, et al. J Biol Chem. 1992; 267: 14109-14117; and Tokunaga F, Nagasawaq K, Miyataq T, Iwanagaqll S. J Biol Chem. 1988; 263: 17471-17481). Comparatively, in a normal fracture site, factor X binds to factor V on the platelet membrane, which accelerates the generation of thrombin many thousand-fold—a mechanism that leads to the formation of a mature clot that stabilizes the primary hemostatic plug (Probst A, Spiegel H-U. J Investig Surg. 1997; 10: 77-86). The aforementioned suggests that pro-coagulating factors isolated from snake venoms should be suitable as a more natural clotting agent to alter the structural properties of blood clots. In fact, snake venom proteins have been important in elucidating the complex physiological mechanisms that govern the coagulation cascade and determine platelet function. Moreover, they have been instrumental in unraveling the structural and functional relationships of human clotting factors and platelet glycoproteins, because of their potency, selectivity, and high biological efficacy (Hong T-T, Huang J, Lucchesi B R. Am J Physiol Heart Circ Physiol. 2006; 290: H959-67; Han S M, Weaver F A, Comerota A J, Perler B A, Joing M. J Vasc Surg. 2010; 51: 600-9; Sanchez E F, Bush L R, Swenson S, Markland F S. Thromb Res. 1997; 87: 289-302; Swenson S, Bush L R, Markland F S. Arch Biochem Biophys. 2000; 384: 227-37; and Shah A R, Scher L. IDrugs. 2007; 10: 329-35). As a result, several active venom compounds have been identified, isolated, characterized and purified, and are currently used for both diagnostic and medicinal purposes (King G F. Expert Opin Biol Ther. 2011; 11:1469-84; and Butler M S. Nat Prod Rep. 2008; 25: 475-516). As described herein, are compositions comprising snake venom coagulating enzymes (SVCE) as an alternative clotting agent, as opposed to the addition of thrombin, which has been shown to have side effects when used clinically (Oryan A, Alidadi S, Moshiri A. Expert Opin Biol Ther. 2016; 16: 213-32). Specific SVCEs have been shown to be catalytically more active than their mammalian counterparts, and are known to be more heat stable and more resistant to proteolysis due to the presence of additional disulfide bridges (Kang T S, Georgieva D, Genov N, Murakami M T, Sinha M, Kumar R P, et al. FEBS J. 2011; 278: 4544-76). Disclosed herein are biomimetic scaffolds and methods demonstrating the ability of snake venom enzymes to alter the properties of hematoma, and their ability to heal large segmental defects using a rat model.

The coagulating factors from snake venoms were favored for several reasons, for instance, they do not affect other clotting factors, and the molecule's small size makes it less likely to be recognized by the body's immune system when used, and are therefore less likely to be attacked. Additionally, the molecules are selective when attaching to their targets within the body, which minimizes the potential for unwanted side effects. Ecarin was specifically selected because it does not require co-factors for activation.

This disclosure is important for the development of new and improved treatment strategies to enhance the healing of bone injuries, increase quality of life, reduce high treatment costs, and decrease the rate of limb amputations, both in the in civilian population as well as amongst military personnel, as a result of severe trauma and battlefield injuries. The results disclosed herein can provide the requirements for an ex vivo produced hematoma with properties that increase the effectiveness of bone healing by mimicking the intrinsic structural and biological properties of naturally healing fracture hematoma. This disclosure is also important for the development of an ex vivo hematoma that acts as a growth factor reservoir that is osteoinductive and osteoconductive, and that can be combined with a scaffold to form a biomimetic scaffold, to enhance the healing of large segmental bone defects, sub-critical size defects as well as for treatment of non-healing fractures (delayed unions or non-unions). Disclosed are compositions that combine an ex vivo hematoma with, for example, an osteoconductive scaffold that can be designed with mechanical properties suitable for bone repair applications. For example, these scaffolds can be used to regenerate bone across significant distances and control resulting bone density, structure and scaffold remodeling rates. In some aspects, the scaffold can be a three-dimensionally printed scaffold that can be used to replace current bone grafting techniques and bone graft substitutes.

Rapid product translation can be expected, as well as an on-demand application, by simply mixing the required volume of whole blood with a specific concentration of the snake venom coagulating enzyme in the operating room, without highly specialized equipment in a civilian or military clinical setting. The results described here can also benefit biomaterials researchers, by incorporating conformed structural properties of hematoma into the design of biomaterial scaffolds to improve the capability of the poorly performing scaffolds currently used in regenerative medicine to aid in bone repair. Furthermore, pro-coagulating factors isolated from snake venom might be used as a suitable clotting agent to alter the structural properties of blood clots as well as be used to stop uncontrolled bleeding in a hospital setting and on the battlefield. Also disclosed herein is a product that could stop bleeding within seconds that is easy to carry, has a long shelf life, is resorbable or easy to remove, and is inexpensive. Because these snakes evolved to kill their prey by causing instant, massive coagulopathy, they have developed highly specific biological agents that turn blood into gelatin. However, when properly isolated and carefully prepared under controlled conditions, the same coagulating factors, such as ecarin, can instead be used to help save lives. By immediately controlling blood loss, this remarkable property can limit further blood loss in either civilian patients or wounded soldiers on the battlefield.

Disclosed herein are biocompatible snake venom-induced ex vivo hematomas for healing large bone defects. Also disclosed herein are methods for processing and manipulating ex vivo hematomas (blood clots) in specific ways that modifies its ultrastructural characteristics, thereby altering its behavior in various clinical situations. Disclosed herein are methods of treating whole blood or blood products with an agent that alters its structural conformation and changes its biological activity so that it can be used for the treatment of several different medical conditions.

Disclosed herein are compositions, biomimetic scaffolds, and methods that can be used to improve the regeneration and repair of large bone defects, sub-critical size defects as well as for the treatment of non-healing fractures (delayed unions or non-unions). Disclosed herein are organized blood clots (i.e., ex vivo hematomas that can be combined with a scaffold to form a biomimetic scaffold) that can be used to enhance bone healing by serving as a temporary reservoir for the continuous release of important growth factors, and by providing adequate space will simultaneously assist with cell infiltration, proliferation, and differentiation. Disclosed herein are biomimetic scaffolds, compositions, and methods for enhancing the repair process that involves producing a blood clot (ex vivo hematoma) that mimics a naturally healing fracture hematoma. The concept is that the structural properties of an ex vivo product, an ex vivo hematoma, that can be altered using a coagulating factor derived from snake venom, ecarin, to mimic an intrinsic fracture hematoma which, when combined with a bone substitute and/or a scaffold and implanted into a bone defect, will enhance and accelerate bone healing. A coagulating factor derived from snake venom, ecarin, can also be used to stop bleeding. In some aspects, the composition can be formulated as a powder, liquid or spray.

Disclosed herein are compositions comprising whole blood, ecarin and BMP-2. In some aspects, ecarin can be present at a specific concentration. In some aspects, a low or markedly reduced dose of BMP-2 can be used in the compositions disclosed herein. In some aspects, the disclosed compositions can be used to enhance bone defect healing. In some aspects, the disclosed compositions can be formulated as a liquid or a gel.

The type of healing described herein is superior to the healing provided by a commercially available product sold by Medtronic, which uses rhBMP-2 delivered on an absorbable collagen sponge (Infuse™). The composition or product described herein requires a significantly lower dose of BMP-2 (e.g., rhBMP-2) when combined with a scaffold to initiate bone healing. In some aspects, the dose of BMP-2 can be 1, 2, 3, 4, 5, 6, 7, 8, 9, 10, 15, 20 or more times lower than any commercially available product or compositions.

As disclosed herein, BMP-2 belongs to a family of bone morphogenetic proteins (BMPs). These proteins are bone growth factors with the ability to induce endochondral bone neoformation. In some aspects, BMP-2 can be recombinant BMP-2. Methods of producing recombinant BMP-2 are known in the art, and for example, can be found in U.S. Pat. No. 7,354,901 which is hereby incorporated by reference herein.

Also disclosed herein are biomimetic scaffolds that can be prepared on demand by pouring/submerging a 3D printed cage (e.g., implant) or mixing with various bone substitutes, into a solution of components that can be used to prepare an ex vivo hematoma (e.g., blood products and coagulants at specific concentrations (calcium and thrombin or ecarin, and with or without growth factors)), and allowing it to congeal for 15-20 min, and then implanting the biomimetic scaffold into the intended treatment/injury site. The biomimetic scaffold comprising a scaffold and an ex vivo hematoma as described herein is currently the only known composition capable of effectively delivering such an extremely low dose of rhBMP-2 with such efficiency, consistently and robustly initiating the fracture healing cascade to successfully repair large critical-sized bone defects. Moreover, the procurement and preparation of the ex vivo hematoma can be done on demand intra-operatively, very quickly, and reproducibly at a nominal cost.

### Compositions

Disclosed herein are compositions comprising: 1) an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride; and 2) a bone substitute. Also disclosed herein are compositions comprising: A composition comprising: 1) an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride; and 2) a bone substitute. In some aspects, the ex vivo hematomas can comprise: (a) isolated whole blood; (b) sodium citrate; and (c) ecarin; oscutarin and calcium chloride; calcium chloride; thrombin; or thrombin and calcium chloride. In some aspects, the ex vivo hematomas can comprise: (a) platelet rich plasma, plasma, or plasma with red blood cells; and (b) ecarin; oscutarin and calcium chloride; calcium chloride; thrombin; or thrombin and calcium chloride. In some aspects, the ex vivo hematoma can further comprise sodium citrate. In some aspects, the phrase “plasma with red blood cells” means plasma without platelets. In some aspects, the ex vivo hematomas can comprise fibrin fibers having a thickness of at least 150-300 nm±10%. In some aspects, the ecarin; oscutarin and calcium chloride; calcium chloride; thrombin; or thrombin and calcium chloride can result in the formation of one or more fibrin fibers having a thickness of at least 150-300 nm±10%. The term “ex vivo” as used herein refers to a hematoma that can be formed outside of an organism, for example, in an external environment. In some aspects, the ex vivo hematoma can comprise (a) isolated whole blood and sodium citrate platelet rich plasma, plasma alone, plasma with red blood cells (without platelets) or other blood products; and (b) one or more coagulating factors. In some aspects, the ex vivo hematomas can comprise whole blood and one or more coagulating factors.

As used herein, the terms “whole blood” and “blood” are used here to mean blood that can be drawn directly from the body from which none of the components, including plasma or platelets, have been removed. In some aspects, the whole blood or blood can be from the subject or patient that will be the receipt of any of the compositions described herein or any of the ex vivo hematomas described herein. In some aspects, the whole blood or blood can be from a donor subject or patient. Whole blood is made up of red blood cells, white blood cells, platelets and plasma. In some aspects, a fibrin gel can be used in place of whole blood.

One of ordinary skill in the art will appreciate that blood is a specialized body fluid that delivers important substances such as nutrients and oxygen to the cells and transports metabolic waste products away from those same cells. In vertebrates, blood is composed of blood cells suspended in blood plasma. Blood can comprise different components, for example, plasma, red blood cells (erythrocytes), platelets (thrombocytes) and white blood cells (leukocytes). Plasma is the main component, making up about 55%, of blood, consisting of mostly water with ions, proteins, nutrients and wastes. Plasma can contain some of every protein produced by the body. For example, plasma can comprise about 90% water and 10% of a mix of the following: ions (Na⁺, K⁺, Mg⁺², Ca⁺², Cl⁻, HCO₃⁻, HPO₄⁻², SO₄⁻²) (Nezafati et al., 2012); proteins (e.g., mainly albumin-55%, globulin, growth factors, enzymes, hormones, antibodies); clotting factors (Factors I-XIII) (labtestsonline.org.au); sugars (glucose); lipids (cholesterols); minerals (sodium, calcium, magnesium, potassium, iron, zinc, copper, and selenium) (Harrington et al., 2014); waste products; and dissolved gases. Red blood cells (erythrocytes) are responsible for carrying oxygen and carbon dioxide. They are about 7-8 μm in size, contain no mitochondria or nucleus when mature, and have an average life span of 120 days. Women have about 3.6-5.0 million/mm³ red blood cells, and men have about 4.2-5.4 million/mm³ red blood cells. Platelets (thrombocytes) are responsible for blood clotting. A normal platelet count ranges from about 150,000 to 450,000/mm³. White blood cells (WBCs; leukocytes) are part of the immune system and function in immune responses. About 1% of the cells are found in blood. They are larger in size than red blood cells, and contain a normal nucleus and mitochondria. A normal white blood cell count ranges from about 5,000-10,000/mm³. White blood cells can be divided into 5 major types that are further divided into two different groups: Granulocytes: Neutrophils: 60-70% of WBCs or 3,000-7,000/mm³, Eosinophils: 1-3% of WBCs or 50-400/mm³, and Basophils: 0.3-0.5% of WBCs or 25-200/mm³; and Agranulocytes: Lymphocytes: 20-30% of WBCs or 1,000-4,000/mm³, and Monocytes: 3-8% of WBCs or 100-600/mm³.

As used herein, the term “platelet rich plasma” (also known as autologous conditioned plasma) refers to a concentrated form of platelet rich plasma protein that is derived from whole blood. For example, whole blood can be centrifuged to remove red blood cells. In some aspects, the term “blood plasma alone”, “plasma alone” or “plasma” can refer to a yellowish liquid component derived from whole blood that normally holds blood cells in whole blood in suspension. For example, blood plasma can be separated from whole blood by centrifuging blood until the blood cells fall to the bottom of the tube, and then the plasma can be drawn off from the top of the tube. In some aspects, the term “plasma with red blood cells” can refer to “plasma alone” with added red blood cells. For example, red blood cells can be derived by centrifuging whole blood until they fall to the bottom of the tube, and are retrieved after removing plasma, white blood cell and platelets from the top of the tube.

In some aspects, the composition can comprise one or more bone substitutes. In some aspects, the one or more bone substitutes can be derived from biological products, can be a synthetic bone substitute or a combination thereof. Examples of bone substitutes derived from biological products include but are not limited to demineralized bone matrix (DBM), bone morphogenetic proteins (BMPs), hydroxyapatite (HA) and corals, allogeneic cancellous bone chips, or bone marrow aspirate concentrate (BMAC), including bone graft from long bones harvested using the reamer irrigation aspirator (RIA). In some aspects, the one or more bone substitutes can be derived from a biological product, wherein the biological product can be bone morphogenetic proteins (BMPs), platelet derived growth factor (PDGF), vascular endothelial growth factor (VEGF), demineralized bone matrix (DBM), hydroxyapatite (HA), corals, allogeneic cancellous bone chips, or bone marrow aspirate concentrate (BMAC), including bone graft from long bones harvested using the reamer irrigation aspirator (RIA). In some aspects, the bone substitutes can be synthetic bone substitutes. Examples of synthetic bone substitutes can include but not are limited to calcium sulfate, calcium phosphate cements, beta-tri-calcium phosphate (TCP) ceramics, biphasic calcium phosphates (Hydroxyapatite (HA) and Beta-TCP ceramics), bioactive glasses, and polymer-based bone substitutes. Further examples of synthetic bone substitutes include but are not limited to Calcigen® S Calcium Sulfate Bone Void Filler, STIMULAN® Beads, HydroSet Injectable Bone Substitute (calcium phosphate), Ossilix calcium phosphate cement, Syntoss Synthetic Beta-Tricalcium Phosphate Bone Graft Material, CERASORB® Tri-Calcium Phosphate Bone Graft, GL1894P/-20 58S BIOACTIVE GLASS, UniGraft Bioactive Glass 200-600 um, BonAlive (BonAlive Biomaterials Ltd, Finland), Cerament (bone void filler) and Cerament G (Bonesupport Holding AB, Lund Sweden). Examples of polymers include but are not limited to collagen, gelatin, chitosan, and synthetic polymers such as poly(lactic acid) (PLA), poly(glycolic acid) (PGA), polycaprolactone (PCL)—GalaFlex P4HB biopolymer, and the like. In some aspects, the bone substitutes can be available in a variety of forms including but not limited to dry, moldable or injectable forms, and pastes, powders, putty, granules, gels, sponges, or strips. In some aspects, the bone substitute can be a commercially available product. In some aspects, the bone substitute can be demineralized bone matrix (DBX; MTF Biologics, Edison, NJ), RegenaVate DBM, Puros DBM, StaGraft DBM, or FiberStack DBM (Zimmer Biomet; Warsaw, IN). In some aspects, the DBM can be an allograft cancellous or cortical bone that has been decalcified to produce a product of collagen and non-collagenous protein. Examples of DBMs include but are not limited to Grafton DBM (Osteotech, Inc, Eatontown, New Jersey), Allosource (Denver, Colorado), Dynagraft II (Integra LifeSciences, Plainsboro, New Jersey), DBX (Musculoskeletal Transplant Foundation and Synthes, Paoli, Pennsylvania), Osteofil (Medtronic Sofamor Danek, Minneapolis, Minnesota). Examples of corals include but are not limited to animalia, coelenterate, scleractenia, poratidae, porites species, and gonioporas species; each of which can be used for developing coralline hydroxyapatite (CHA) bone substitute. In some aspects, the bone substitute is not BMP, rhBMP-2 or BMP-2.

In some aspects, the ratio of the ex vivo hematoma to bone substitute can be from 1000:1 to 1:1000 or any ratio in between. In some aspects, the ratio of the ex vivo hematoma to bone substitute can be 1:1, 1:2, 1:3, 1:4, 1:5, 1:6, 1:7, 1:8, 1:9, or 1:10. In some aspects, the ratio of the bone substitute to ex vivo hematoma can be 1:1, 1:2, 1:3, 1:4, 1:5, 1:6, 1:7, 1:8, 1:9, or 1:10.

In some aspects, the composition, ex vivo hematoma, bone substitute or biomimetic scaffold can comprise one or more growth factors. In some aspects, the one or more growth factors can be one or more of the bone morphogenetic proteins. Examples of BMPs include but are not limited to BMP-2, BMP-7, BMP-4, BMP-6, BMP-9, and BMP-14 (also known as GDF5). Any BMPs are contemplated, including BMP-1 through BMP-18. In some aspects, the one or more growth factors can be platelet-derived growth factor. In some aspects, the one or more growth factors can be vascular endothelial growth factor. In some aspects, the one or more growth factors can be fibroblast growth factor 2. In some aspects, the one or more growth factors can be one or more of the bone morphogenetic proteins, platelet-derived growth factor, vascular endothelial growth factor, fibroblast growth factor 2, or a combination thereof. In some aspects, the composition, ex vivo hematoma, bone substitute or biomimetic scaffold can further comprise BMP-2. In some aspects, the one or more growth factors can be BMP-2. In some aspects, the one or more growth factors is not BMP, rhBMP-2, BMP-2, BMP-7, BMP-4, BMP-6, BMP-9, or BMP-14.

In some aspects, the whole blood can comprise viable cells. In some aspects, about 50% to 70% of the viable cells of the whole blood remain viable after formation of the ex vivo hematoma. In some aspects, at least 50% of the viable cells of the whole blood remain viable after formation of the hematoma. In some aspects, at least 60% of the viable cells of the whole blood remain viable after formation of the hematoma. In some aspects, at least 70% of the viable cells of the whole blood remain viable after formation of the ex vivo hematoma. In some aspects, at least 80% of the viable cells of the whole blood remain viable after formation of the ex vivo hematoma. In some aspects, at least 90% of the viable cells of the whole blood remain viable after formation of the ex vivo hematoma. In some aspects, more than 90% of the viable cells of the whole blood remain viable after formation of the ex vivo hematoma.

In some aspects, the whole blood can comprise one or more biological factors. In some aspects, the term “biological factors” or “other biological factors” refers to the plasma component of whole blood excluding water. Examples of other biological factors include but are not limited to ions, proteins, clotting factors, sugars, lipids, and minerals.

In some aspects, the one or more biological factors present in the whole blood can be endogenous biological factors. Platelets are present in whole blood. Many growth factors can be found in platelets. Growth factors in platelet-rich plasma platelet α-granules have been shown to contain mitogenic and chemotactic growth factors along with associated healing molecules in an inactive form, which are important in wound healing, including but not limited to platelet-derived growth factor (PDGF), transforming growth factors β1, β2, β3 (TGF-β1, TGF-β2, TGF-β3, platelet-derived angiogenesis factor (PDAF), insulin-like growth factor 1 (IGF-1), platelet factor 4 (PF-4), epidermal growth factor (EGF), epithelial cell grow factor (ECGF), vascular endothelial cell growth factor (VEGF), basic fibroblast growth factor (bFGF) and other cytokines. Additionally, plasma fluid also contains a number of biologically active proteins such as growth factor IGF-I and hepatocyte growth factor (HGF). During normal wound healing, trapped platelets become activated and degranulate, resulting in the release of the α-granule content. Examples of growth factors present in platelets include but are not limited to platelet-derived growth factors, transforming growth factors β1, β2, β3, platelet-derived angiogenesis factor, insulin-like growth factor 1, platelet factor 4, epidermal growth factor, epithelial cell growth factor, vascular endothelial cell growth factor, basic fibroblast growth factor, and others cytokines; as well as platelet-derived endothelial growth factor (PDEGF), interleukin 1, osteocalcin and osteonectin. Growth factors present in plasma fluid include but are not limited to insulin-like growth factor 1, and hepatocyte growth factor.

In some aspects, the ex vivo hematoma can comprise whole blood, ecarin and sodium citrate. In some aspects, the ex vivo hematoma can comprise whole blood, calcium chloride and sodium citrate. In some aspects, the ex vivo hematoma can comprise platelet rich plasma and ecarin. In some aspects, the ex vivo hematoma can comprise platelet rich plasma and calcium chloride. In some aspects, the ex vivo hematoma can comprise whole blood; calcium chloride; or oscutarin and calcium chloride; and sodium citrate. In some aspects, a combination of one of (a) isolated whole blood and sodium citrate; platelet rich plasma, or plasma with red blood cells can be combined with one of (b) ecarin, oscutarin and calcium chloride, or calcium chloride. In some aspects, a combination of one of (a) isolated whole blood and sodium citrate; platelet rich plasma, or plasma with red blood cells can be combined with one of (b) thrombin or thrombin and calcium chloride. In some aspects, any of the ex vivo hematoma combinations described herein can further comprise one or more antibiotics.

In some aspects, the concentration of calcium chloride present in the ex vivo hematoma can be in the range of 1 mM to 20 mM. In some aspects, the concentration of calcium chloride can be 1, 2, 3, 4, 5, 6, 7, 8, 9, 10, 11, 12, 13, 14, 15, 16, 17, 18, 19, 20 mM or any number in between. In some aspects, the concentration of calcium chloride can be about 10 mM.

In some aspects, the concentration of thrombin can be in the range of 0.1 to 1 U/mL. In some aspects, the concentration of thrombin present in the ex vivo hematoma can be 0.05, 0.1, 0.2, 0.3, 0.4, 0.5, 0.6, 0.7, 0.8, 0.9, 1 U/mL or any number in between or higher. In some aspects, the concentration of thrombin present in the ex vivo hematoma can be 0.5 U/mL

In some aspects, the concentration of ecarin present in the ex vivo hematoma can be at least 0.05 U/mL. In some aspects, the concentration of ecarin present in the ex vivo hematoma can be 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 U/mL or any number in between or higher. In some aspects, the concentration of ecarin present in the ex vivo hematoma can be 0.3 U/mL. In some aspects, the concentration of ecarin present in the ex vivo hematoma can be 0.6 U/mL. In some aspects, the concentration of ecarin present in the ex vivo hematoma can be 0.75 U/mL. In some aspects, the concentration of ecarin present in the ex vivo hematoma can be any value (rational or irrational) between 0 and 2.

In some aspects, the ex vivo hematomas, bone substitutes, biomimetic scaffolds or compositions described herein can further comprise BMP-2. In some aspects, the BMP-2 can be a recombinant BMP-2. In some aspects, the recombinant BMP-2 can comprise human BMP-2. In some aspects, the dose of BMP-2 present in the ex vivo hematoma, bone substitute, composition, or biomimetic scaffold can be at least 0.01 mg. In some aspects, the dose of BMP-2 present in the ex vivo hematoma, bone substitute, composition, or biomimetic scaffold can be 0.01 to 5 mg. In some aspects, the dose of BMP-2 present in the ex vivo hematoma, bone substitute, composition, or biomimetic scaffold can be 0.01, 0.2, 0.3, 0.4, 0.5, 0.6, 0.7, 0.8, 0.9, 1.0, 1.5, 2.0, 2.5, 3.0, 3.5, 4.0, 4.5, 5.0 mg or any number in between. In some aspects, recombinant BMP-2 can be used at a dose of about 0.01 mg to about 12 mg. In some aspects, recombinant BMP-2 can be used at a dose of 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 mg or any number in between. In some aspects, recombinant BMP-2 can be used at a dose higher than 12.0 mg. In some aspects, dose of BMP-2 can be about 1 mg to 5 mg. In some aspects, the dose of BMP-2 present in the ex vivo hematoma, bone substitute, composition, or biomimetic scaffold can be at least 0.01 μg. In some aspects, the dose of BMP-2 present in the ex vivo hematoma, bone substitute, composition, or biomimetic scaffold can be 0.01 to 5 μg. In some aspects, the dose of BMP-2 present in the ex vivo hematoma, bone substitute, composition, or biomimetic scaffold can be 0.01, 0.2, 0.3, 0.4, 0.5, 0.6, 0.7, 0.8, 0.9, 1.0, 1.5, 2.0, 2.5, 3.0, 3.5, 4.0, 4.5, 5.0 μg or any number in between. In some aspects, recombinant BMP-2 can be used at a dose of about 0.01 μg to about 12 μg. In some aspects, recombinant BMP-2 can be used at a dose of 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 μg or any number in between. In some aspects, recombinant BMP-2 can be used at a dose higher than 12.0 μg. In some aspects, dose of BMP-2 can be about 1 μg to 5 μg. In some aspects, the dose of BMP-2 present in the ex vivo hematoma, bone substitute, composition, or biomimetic scaffold can be between 0.3 and 0.4 μg. In some aspects, the dose of BMP-2 present in the ex vivo hematoma, bone substitute, composition, or biomimetic scaffold can be lower than the standard dose. In some aspects, the dose of BMP-2 present in the ex vivo hematoma, bone substitute, composition, or biomimetic scaffold can be 10-50 times lower than the standard dose or the lowest effective dose of BMP-2/ACS.

In some aspects, the amount of the ecarin present in the ex vivo hematoma can be at least 0.05 U/mL; and the amount of BMP-2 present in the ex vivo hematoma can be at least 0.01 mg.

In some aspects, the amount of the ecarin present in the ex vivo hematoma can be at least 0.05 U/mL; and the amount of BMP-2 present in the ex vivo hematoma can be at least 0.01 μg.

In some aspects, the concentration of sodium citrate can be about 3.2 to 4 mg/ml. In some aspects, the solution is about 3.2 to 4% (weight/volume) sodium citrate, in which one part of this solution can then be mixed with nine parts whole blood.

In some aspects, the ex vivo hematomas or compositions described herein can further comprise one or more therapeutic agents. In some aspects, the therapeutic agent can be a growth factor. In some aspects, the therapeutic agent can be BMP-2. In some aspects, the therapeutic agent can be recombinant BMP-2. In some aspects, the therapeutic agent can be stem cells or pre-differentiated stem cells, including but not limited to mesenchymal stem cells, adipose stem cells, and induced pluripotent stem cells. In some aspects, the therapeutic agent can be ecarin.

In some aspects, the ex vivo hematoma, biomimetic scaffolds or compositions disclosed herein can be formulated as a liquid or a gel. In some aspects, the ex vivo hematoma, biomimetic scaffolds or compositions disclosed herein can be formulated as a paste or a putty. In some aspects, the ex vivo hematomas can be formulated as a lyophilized or powder form. Said lyophilized or powder forms can make the ex vivo hematoma more stable for storage. In some aspects, the ex vivo hematoma, biomimetic scaffolds or compositions disclosed herein can be formulated as a liquid, a gel, a powder, granules, a paste or a putty. In some aspects, the composition can be formulated as a lyophilized or powder form. Said lyophilized or powder forms can make the ex vivo hematoma, biomimetic scaffolds or compositions disclosed herein more stable for storage. In some aspects, the growth factors (BMPs and others), coagulating factors (ecarin, calcium chloride, etc.) and sodium citrate can be available as a lyophilized or powder forms. In some aspects, compounds used for making the ex vivo hematomas disclosed herein can be dissolved in sterile distilled water prior to mixing with whole blood or other blood products (e.g., PRP, plasma, etc.). The diluent is sterile distilled water. No additional components are needed for preparation or storage. Whole blood (or other blood products) can be drawn from a patient before (e.g., immediately before) the surgery, and citrated to prevent clotting. In some aspects, donor blood can be used, for example, for patients with a blood disorder or diseases including but not limited to anemia, hemophilia, leukemia, HIV, etc. The remainder of the components of the ex vivo hematoma do not require any additional stabilizers for storage. For example, BMP-2 is commercially available in a bottle, ready to use; and CaCl₂) is available in a powder form, and in some cases may already be dissolved in sterile distilled water (it is very stable after it is dissolved). Both BMP-2 and CaCl₂) can be stored at room temperature. Ecarin is available in a lyophilized form (freeze-dried), stored at −20° C., and can be dissolved in sterile distilled water prior to use. The ex vivo hematoma can be prepared relatively simply, using the components described herein in amounts based on the volume of the defect to fill. For this, after the components are prepared, they can be mixed together in a tube/mold. Generally, the ex vivo hematoma will form in about 30 to 45 minutes and then can be inserted (or implanted) into the bone defect. In some aspects, the ex vivo hematomas described herein can be stored, for example, using a “smart storage system” that uses radio-frequency identification-based system (e.g., Smartstorage™) which involves a near real-time tissue tracking system that can streamline inventory management including keeping accurate usage history and temperature logs.

### Scaffolds

Disclosed herein are biomimetic scaffolds. The biomimetic scaffolds can comprise any of the scaffolds described herein and any of the ex vivo hematomas described herein.

Disclosed herein are biomimetic scaffolds comprising a scaffold and an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride. Disclosed herein are biomimetic scaffolds comprising a scaffold and an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride. In some aspects, the ex vivo hematoma can comprise fibrin fibers having a thickness of at least 150-300 nm±10%. In some aspects, the biomimetic scaffold further comprises one or more bone substitutes. In some aspects, the ex vivo hematoma of the biomimetic scaffold comprises one or more bone substitutes.

In some aspects, the ex vivo hematomas described herein can further be combined with a carrier such as a scaffold. For example, the carrier can be a biodegradable biomaterial scaffold (e.g., silk fibroin scaffolds, poly(lactide-co-glycolide (PLGA), or other similar resorbable products or materials). Such carrier can be used to provide additional mechanical support of the ex vivo hematoma. Examples of scaffolds for use in the disclosed biomimetic scaffolds, include, but are not limited to a biocompatible scaffold, an osteoconductive scaffold, a three-dimensionally printed scaffold, collagen, absorbable collagen scaffold (collagen type 1 bovine or porcine), collagen bovine membrane, or collagens derived from other mammalian or non-mammalian (such as marine) sources, chitins, bioabsorbable polymers such PLA, or nonabsorbable polymers such as PEEK, or titanium or other biocompatible metallic alloys such as resorbable magnesium (such as magnesium calcium alloys) or a three-dimensionally printed scaffold.

In some aspects, the scaffolds that can be combined with the ex vivo hematomas described herein can be biocompatible and biodegradable. For instance, the scaffold should elicit a negligible immune reaction in order to prevent it from causing such a severe inflammatory response that it might reduce healing or cause rejection by the body. In some aspects, the scaffold is amendable to cells adhering to it and so that the cells can function normally and migrate to the surface and eventually through the scaffold to proliferate. The scaffold disclosed herein can also be biodegradable to that cells can produce their own extracellular matrix. The by-products of this degradation can also be non-toxic and able to exit the body without interference from other organs.

In some aspects, the scaffold can have mechanical properties that are consistent with the anatomical site into which it is to be implanted and strong enough to allow surgical handling during implantation. Further, the scaffold can be sufficient to allow cell infiltration and vascularization. In designing the appropriate scaffold, it is important to consider the architecture of the scaffold. For instance, the scaffolds can be have an interconnected pore structure and high porosity to ensure cellular penetration and adequate diffusion of nutrients to cells. A porous interconnected structure is important to allow diffusion of waste products out of the scaffold, and the products of scaffold degradation should be able to exit the body without interference with other organs and surrounding tissues.

In some aspects, the scaffold can be a three-dimensionally printed scaffold. In some aspects, the three-dimensionally printed scaffold can be custom produced.

The three-dimensional scaffold can be produced using a 3-D printing technique referred to as robotic deposition or direct write (DW) technology. This technique uses a computer controlled printing process and colloidal inks to form three-dimensional structures. These structures can form on the self-components or can be custom formed for filling individual bone defects from tomographic data (X-ray, sonographic or MRI).

Ink fabrication and the printing system itself uses water-based rheologically controlled inks that become solid as they leave the print nozzle. These inks consist of finely controlled ceramic particles in a water-based slurry containing organic chemicals that control the handling characteristics of the colloidal ink. This allows 3-D lattice-like structures to be printed, in layers, without or with minimal sag of unsupported structural elements.

Using this system, the elements of the first layer can be printed by forcing the ink through a small (^{˜}50-400 μm diameter) nozzle onto a support plate, using the x and y coordinate control system of an x-y-z control gantry system. Then the z control system is used to move the nozzle up slightly less than 1 nozzle diameter. Then the next layer is printed over the first layer. This is continued layer-by-layer until the entire 3-D structure is finished.

The entire structure can be printed in an oil bath to prevent drying. The system can have 3 nozzles and ink reservoirs so that up to three materials can be used to print a single structure. Fugitive inks, inks consisting entirely of material that burn off during firing, can also be used as part of the printing process. These can be used to print support structures for complex parts requiring temporary supports.

The resulting structures are then removed from the oil bath, dried, and fired in a programmable furnace to produce the final ceramic structure. Firing is currently done at approximately 1100° C. for about four hours, which substantially burns off the organic components, sintering the ceramic particles together into a solid structure. This may cause a small amount of predictable shrinkage that can be calculated into the printing process to produce precise and predictable structures.

The print nozzles can be routinely cylindrical producing cylindrical rod printed structures. However, nozzles can be made that are shaped to produce non-cylindrical structures or structures with surface striations of sizes designed to control cell migration, growth, and differentiation.

In some aspects, a variety of biomaterials can be used to fabricate the scaffolds disclosed herein. In some aspects, the biomaterials can be ceramics, synthetic polymers and/or natural polymers or combinations thereof. Examples of ceramics include but are not limited hydroxyapatite (HA) and tri-calcium phosphate (TCP). Examples of synthetic polymers include but are not limited to polystyrene, poly-l-lactic acid (PLLA), polyglycolic acid (PGA) and poly-dl-lactic-co-glycolic acid (PLGA). Examples of natural polymers include but are not limited to collagen, various proteoglycans, alginate-based substrates and chitosan. The advantage of using natural polymers is that they are biologically active and typically promote excellent cell adhesion and growth. Furthermore, they are also biodegradable and so allow host cells, over time, to produce their own extracellular matrix and replace the degraded scaffold. In some aspects, the scaffold can be made of a combination of biomaterials. In some aspects, collagen can be combined with a polysaccharide (e.g., glycosaminoglycan). In some aspects, the scaffolds can be prepared by using chemical cross-linking methods.

In some aspects, the compositions or biomimetic scaffolds comprising the scaffold and ex vivo hematoma can be formulated for local administration. In some aspects, the compositions (e.g., liquid form) or ex vivo hematomas (e.g., gel form) disclosed herein can be combined with any of the carriers or scaffolds disclosed herein and administered locally or implanted surgically or injected percutaneously. In some aspects, the liquid formulation can be delivered through a syringe to the scaffold. In some aspects, the gel formulation can be implanted into the bone defect site. The gel formulation can be prepared using a mold outside of the body that corresponds to the bone size and shape for implantation into the bone defect site. In some aspects, the formulation can be in an intermediate form, between liquid and gel. In some aspects, an intermediate formulation can be applied to a solid scaffold or carrier to bridge gaps that may be present in the solid scaffold itself (e.g., large gaps) while also providing mechanical support independently. Examples of a solid scaffold includes but is not limited to titanium cages or other porous metallic implants. Such scaffolds can be used to reconstruct a skeletal defect or to achieve spinal fusion. The formulations disclosed herein can be used to augment healing when PEEK spinal cages are used for interbody spinal fusions, considering PEEK is itself biologically inert and has no intrinsic bone healing capacity. Alternatively, any of the formulations disclosed herein can be infused or applied topically to resorbable scaffolds as may be used to reconstruct skeletal defects, segmental or subsegmental. When used in conjunction with either metallic porous implants or resorbable scaffolds, this would also include opening wedge osteotomies (of the femur, tibia, or other long bones), distraction arthrodesis sites, and bone defects related to arthroplasty. Furthermore, any of the biomimetic scaffolds, compositions and ex vivo hematoma formulations disclosed herein can be applied in the same fashion to other arthrodesis sites with bone defects such as the ankle, knee, wrist, shoulder, hip, or other smaller joints, including but not limited to the Lisfranc joint, smaller joints in the hand, wrist, or foot, and extends to include applications to fill bone defects created when harvesting bone grafts for transposition to a secondary anatomic location.

### Devices

Disclosed herein are multi-compartment devices for delivering any of the compositions and/or ex vivo hematomas described herein. In some aspects, the multi-compartment device can comprise two or more chambers or two or more syringes can be used to deliver components of the ex vivo hematoma and the bone substitutes separately. In some aspects, a first chamber or syringe can comprise the coagulants (e.g., calcium and thrombin, or ecarin) at pre-determined concentrations. In some aspects, a second chamber or syringe can comprise whole blood alone. In some aspects, a second chamber or syringe can comprise whole blood in combination with exogenous growth factors (e.g., BMP2, PDGF, VEGF). In some aspects, a second chamber or syringe can comprise whole blood in combination with bone substitutes (e.g., DBM, allogeneic cancellous bone chips). In some aspects, a second chamber or syringe can comprise whole blood in combination with exogenous growth factors (e.g., BMP2, PDGF, VEGF) bone substitutes. In some aspects, a third chamber or syringe can comprise exogenous growth factors and additional bone substitutes. In some aspects, a third chamber or syringe can comprise exogenous growth factors. In some aspects, a third chamber or syringe can comprise one or more bone substitutes. In some aspects, a fourth chamber or syringe can comprise one or more bone substitutes.

### Methods

Disclosed herein are methods of promoting bone healing. Also disclosed herein are methods of producing bone replacement material. Further disclosed herein are methods of producing implants or biomimetic scaffolds or any of the compositions described herein. In some aspects, the compositions comprising the ex vivo hematoma can also serve as a scaffold. It some aspects, the compositions comprising the ex vivo hematoma can be combined with a scaffold to form or create a biomimetic scaffold. In some aspects, the methods disclosed herein can be combined. Disclosed herein are methods of promoting bone healing, producing bone replacement material, producing implants, compositions comprising ex vivo hematomas, biomimetic scaffolds or a combination thereof.

Disclosed herein are methods of promoting bone healing or producing bone replacement material or implants. In some aspects, the methods comprise administering to a subject in need thereof a therapeutically effective amount of any of the compositions disclosed herein. In some aspects, the methods can comprise administering to a subject in need thereof a therapeutically effective amount of a composition comprising the ex vivo hematoma disclosed herein and one or more bone substitutes. In some aspects, the methods comprise implanting any of the biomimetic scaffold described herein into a site of interest in the subject. In some aspects, the biomimetic scaffold can further comprise one or more bone substitutes.

In some aspects, the ex vivo hematomas can comprise: (a) isolated whole blood; (b) sodium citrate; and (c) ecarin; oscutarin and calcium chloride; calcium chloride; thrombin; or thrombin and calcium chloride. In some aspects, the ex vivo hematomas can comprise: (a) platelet rich plasma, plasma, or plasma with red blood cells; and (b) ecarin; oscutarin and calcium chloride; calcium chloride; thrombin; or thrombin and calcium chloride. In some aspects, the ex vivo hematoma can further comprise sodium citrate. In some aspects, the ex vivo hematomas can comprise fibrin fibers having a thickness of at least 150-300 nm±10%. In some aspects, the ecarin; oscutarin and calcium chloride; calcium chloride; thrombin; or thrombin and calcium chloride can result in the formation of one or more fibrin fibers having a thickness of at least 150-300 nm±10%. In some aspects, the ex vivo hematoma can comprise (a) isolated whole blood and sodium citrate platelet rich plasma, plasma alone, plasma with red blood cells (without platelets) or other blood products; and (b) one or more coagulating factors. In some aspects, the ex vivo hematomas can comprise whole blood and one or more coagulating factors. In some aspects, the whole blood can comprise one or more viable cells. In some aspects, the whole blood can comprise one or more biological factors. In some aspects, the ex vivo hematoma can comprise whole blood, ecarin, and sodium citrate. In some aspects, the ex vivo hematoma can comprise whole blood, calcium chloride, and sodium citrate. In some aspects, the ex vivo hematoma can comprise platelet rich plasma and ecarin. In some aspects, the ex vivo hematoma can comprise platelet rich plasma and calcium chloride. In some aspects, the ex vivo hematoma can comprise plasma and ecarin. In some aspects, the ex vivo hematoma can comprise plasma and calcium chloride. In some aspects, the ex vivo hematoma can comprise plasma with red blood cells and ecarin. In some aspects, the ex vivo hematoma can comprise plasma with red blood cells and calcium chloride. In some aspects, the ex vivo hematoma can comprise plasma with oscutarin and calcium chloride. In some aspects, the ex vivo hematoma can comprise plasma with thrombin and calcium chloride. In some aspects, the ex vivo hematoma can further comprise bone morphogenetic protein 2 (BMP-2). In some aspects, the BMP-2 can be recombinant BMP-2. In some aspects, the recombinant BMP-2 can comprise human BMP-2. In some aspects, the composition can further comprise one or more growth factors, one or more platelets, and one or more cells. In some aspects, the composition can be formulated as a clot or as a scaffold. In some aspects, the scaffold can be chemotactic. In some aspects, the scaffold can attract endogenous growth factors conducive to bone healing.

In some aspects, the one or more bone substitutes can be derived from biological products, be a synthetic bone substitute or a combination thereof. Examples of bone substitutes derived from biological products include but are not limited to demineralized bone matrix (DBM), bone morphogenetic proteins (BMPs), hydroxyapatite (HA) and corals, allogeneic cancellous bone chips, or bone marrow aspirate concentrate (BMAC), including bone graft from long bones harvested using the reamer irrigation aspirator (RIA). In some aspects, the one or more bone substitutes can be derived from a biological product, wherein the biological product can be bone morphogenetic proteins (BMPs), platelet derived growth factor (PDGF), vascular endothelial growth factor (VEGF), demineralized bone matrix (DBM), hydroxyapatite (HA), corals, allogeneic cancellous bone chips, or bone marrow aspirate concentrate (BMAC), including bone graft from long bones harvested using the reamer irrigation aspirator (RIA). In some aspects, the bone substitutes can be synthetic bone substitutes. Examples of synthetic bone substitutes can include but not are limited to calcium sulfate, calcium phosphate cements, beta-tri-calcium phosphate (TCP) ceramics, biphasic calcium phosphates (Hydroxyapatite (HA) and Beta-TCP ceramics), bioactive glasses, and polymer-based bone substitutes. Further examples of synthetic bone substitutes include but are not limited to Calcigen® S Calcium Sulfate Bone Void Filler, STIMULAN® Beads, HydroSet Injectable Bone Substitute (calcium phosphate), Ossilix calcium phosphate cement, Syntoss Synthetic Beta-Tricalcium Phosphate Bone Graft Material, CERASORB® Tri-Calcium Phosphate Bone Graft, GL1894P/-20 58S BIOACTIVE GLASS, UniGraft Bioactive Glass 200-600 um, BonAlive (BonAlive Biomaterials Ltd, Finland), Cerament (bone void filler) and Cerament G (Bonesupport Holding AB, Lund Sweden). Examples of polymers include but are not limited to collagen, gelatin, chitosan, and synthetic polymers such as poly(lactic acid) (PLA), poly(glycolic acid) (PGA), polycaprolactone (PCL)—GalaFlex P4HB biopolymer, and the like. In some aspects, the bone substitutes can be available in a variety of forms including but not limited to dry, moldable or injectable forms, and pastes, powders, putty, granules, gels, sponges, or strips. In some aspects, the bone substitute can be a commercially available product. In some aspects, the bone substitute can be demineralized bone matrix (DBX; MTF Biologics, Edison, NJ), RegenaVate DBM, Puros DBM, StaGraft DBM, or FiberStack DBM (Zimmer Biomet; Warsaw, IN). In some aspects, the DBM can be an allograft cancellous or cortical bone that has been decalcified to produce a product of collagen and non-collagenous protein. Examples of DBMs include but are not limited to Grafton DBM (Osteotech, Inc, Eatontown, New Jersey), Allosource (Denver, Colorado), Dynagraft II (Integra LifeSciences, Plainsboro, New Jersey), DBX (Musculoskeletal Transplant Foundation and Synthes, Paoli, Pennsylvania), Osteofil (Medtronic Sofamor Danek, Minneapolis, Minnesota). Examples of corals include but are not limited to animalia, coelenterate, scleractenia, poratidae, porites species, and gonioporas species; each of which can be used for developing coralline hydroxyapatite (CHA) bone substitute. In some aspects, the bone substitute is not BMP, rhBMP-2 or BMP-2.

In some aspects, the subject can be a human. In some aspects, the subject has a skeletal defect. In some aspects, the skeletal defect can be a large segmental bone defect. In some aspects, the skeletal defect can be a small segmental bone defect. In some aspects, the skeletal defect can be independent of the size or volume of the defect regardless of whether the defect is complete or incomplete. In some aspects, the subject has a dental bone defect.

In some aspects, the subject has one or more bone fractures. In some aspects, the subject has one or more bone injuries.

In some aspects, the composition can be formulated as a clot or a scaffold. In some aspects, the composition can be formulated for local administration and combined with any of the scaffolds disclosed herein. In some aspects, the composition can be administered locally via a carrier or a scaffold. In some aspects, the composition can be administered locally without a carrier or a scaffold. In some aspects, the composition can be implanted or delivered percutaneously. In some aspects, the composition can be implanted. In some aspects, the composition can be implanted either directly or indirectly. In some aspects, the composition can be delivered by a surgeon or by any autonomous or semi-autonomous delivery device acting on behalf of a human or robotic/semi-autonomous agent. In some aspects, the composition can be delivered percutaneously.

Disclosed herein are methods of constructing an implant. In some aspects, the methods can comprise: a) dimensioning a depot implant in at least one of a shape and a size that facilitates implantation of the depot implant into a bone defect; and b) structuring the depot implant to have a scaffold by introducing: (i) isolated whole blood and sodium citrate; or platelet rich plasma, plasma, or plasma with red blood cells; (ii) ecarin; oscutarin and calcium chloride; calcium chloride; thrombin; or thrombin and calcium chloride; and (iii) a bone substitute to create the scaffold. In some aspects, the scaffold can have a porosity of 55 to 75%. In some aspects, the scaffold can comprise fibrin fibers having a thickness of at least 150-300 nm±10%. In some aspects, the shape of the depot implant can be that of a cylinder or a sphere. In some aspects, the scaffold can be constructed as a clot. In some aspects, one or more growth factors can be introduced into the scaffold. In some aspects, the one or more growth factors can be bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof. In some aspects, the BMP-2 can be introduced into the scaffold. In some aspects, the amount of ecarin present in the scaffold can be at least 0.05 U/mL; and the amount of BMP-2 present in the scaffold can be at least 0.01 mg. In some aspects, the ratio of the ex vivo hematoma to bone substitute can be from 1000:1 to 1:1000. In some aspects, the scaffold can resemble the size and shape of a given bone defect. In some aspects, the scaffold can be chemotactic. In some aspects, the scaffold can comprise viable blood cells and appropriate biological factors. In some aspects, the bone substitute can be demineralized bone matrix. In some aspects, the bone substitute can be derived from a biological product, a synthetic bone substitute or a combination thereof. In some aspects, the biological product can be a demineralized bone matrix, hydroxyapatite, or a coral. In some aspects, the synthetic bone substitute can be calcium sulfate, a calcium phosphate cement, β-tri-calcium phosphate ceramics, bioactive glasses, or a polymer. In some aspects, the one or more growth factors is not BMP, rhBMP-2, BMP-2, BMP-7, BMP-4, BMP-6, BMP-9, or BMP-14.

Also, disclosed herein are methods of constructing a biomimetic scaffold. In some aspects, the methods can comprise: a) dimensioning a scaffold in at least one of a shape and a size that facilitates implantation of the scaffold into a bone defect; and b) combining the scaffold in a) with an ex vivo hematoma comprising: (i) isolated whole blood and sodium citrate; or platelet rich plasma, plasma, or plasma with red blood cells; and (ii) ecarin; oscutarin and calcium chloride; calcium chloride; thrombin; or thrombin and calcium chloride to create the biomimetic scaffold. In some aspects, the scaffold can have a porosity of 55 to 75%. In some aspects, the scaffold can comprise fibrin fibers having a thickness of at least 150-300 nm±10%. In some aspects, the shape of the scaffold can be that of a cylinder or a sphere. In some aspects, the shape of the scaffold can be that any and all other geometric shape or shapes that could theoretically occupy a discrete subset or volume within Euclidean space. In some aspects, the scaffold can be collagen, chitins, bioabsorbable polymers, nonabsorbable polymers such as PEEK, or titanium or a metallic alloy. In some aspects, one or more growth factors can be introduced into the scaffold or ex vivo hematoma. In some aspects, the one or more growth factors can be bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof. In some aspects, the BMP-2 can be introduced into the scaffold or ex vivo hematoma. In some aspects, the amount of ecarin present in the scaffold or ex vivo hematoma can be at least 0.05 U/mL; and the amount of BMP-2 present in the scaffold or ex vivo hematoma can be at least 0.01 mg. In some aspects, the ratio of the ex vivo hematoma to bone substitute can be from 1000:1 to 1:1000. In some aspects, the ex vivo hematoma can comprise viable blood cells and appropriate biological factors. In some aspects, the bone substitute can be demineralized bone matrix. In some aspects, the bone substitute can be derived from a biological product, a synthetic bone substitute or a combination thereof. In some aspects, the biological product can be a demineralized bone matrix, hydroxyapatite, or a coral. In some aspects, the synthetic bone substitute can be calcium sulfate, a calcium phosphate cement, 0-tri-calcium phosphate ceramics, bioactive glasses, or a polymer. In some aspects, the scaffold can resemble the size and shape of a given bone defect. In some aspects, the scaffold can be chemotactic. Additionally, the scaffold can be biodegradable such that it degrades without the necessity of surgical removal. In some aspects, the one or more growth factors is not BMP, rhBMP-2, BMP-2, BMP-7, BMP-4, BMP-6, BMP-9, or BMP-14.

In some aspects, the composition can be implanted as all or part of a biomimetic scaffold. In some aspects, the composition can be injected with a syringe into a carrier or scaffold. In some aspects, the amount of the ecarin present in the composition can be at least 0.05 U/mL; and the amount of BMP-2 present in the composition can be at least 0.01-5 mg or any amount in between. In some aspects, the amount of the ecarin present in the composition can be at least 0.05 U/mL and the amount of BMP-2 present in the composition can be at least 0.01-1 μg or any amount in between. In some aspects, the ratio of the ex vivo hematoma to bone substitute can be from 1000:1 to 1:1000 or any ratio in between.

In some aspects, the treatment regimen can be a standard treatment regimen for treating any bone defect. Briefly, the defect wound can be debrided, fixed with an internal plate, external fixator or intramedullary nail. The compositions; compositions and scaffolds; and biomimetic scaffolds and ex vivo hematomas described herein can then be inserted as a unit into the skeletal defect before closing the wound. In some aspects, the components of the compositions; the components of the compositions and the scaffolds; and biomimetic scaffolds and the components of the ex vivo hematomas described herein can then be inserted as separately into the skeletal defect before closing the wound. For example, a multi-compartment device comprising two or more chambers or two or more syringes can be used to deliver components of the ex vivo hematoma and the bone substitutes separately. In some aspects, a first chamber or syringe can comprise the coagulants (e.g., calcium and thrombin, or ecarin) at pre-determined concentrations. In some aspects, a second chamber or syringe can comprise whole blood alone. In some aspects, a second chamber or syringe can comprise whole blood in combination with exogenous growth factors (e.g., BMP2, PDGF, VEGF). In some aspects, a second chamber or syringe can comprise whole blood in combination with bone substitutes (e.g., DBM, allogeneic cancellous bone chips). In some aspects, a second chamber or syringe can comprise whole blood in combination with exogenous growth factors (e.g., BMP2, PDGF, VEGF) bone substitutes. In some aspects, a third chamber or syringe can comprise exogenous growth factors and additional bone substitutes. In some aspects, a third chamber or syringe can comprise exogenous growth factors. In some aspects, a third chamber or syringe can comprise one or more bone substitutes. In some aspects, a fourth chamber or syringe can comprise one or more bone substitutes. In some aspects, the one or more growth factors is not BMP, rhBMP-2, BMP-2, BMP-7, BMP-4, BMP-6, BMP-9, or BMP-14.

The treatment regimen can be consistent and invariant provided there is no infection present and the defect is otherwise ready for definitive treatment. The compositions or biomimetic scaffold (or implant) disclosed herein can be inserted into bone areas by entering the body through the skin or through a body cavity or an anatomical opening to minimize any additional damage to nearby structures. Selection of the type, including size and shape, of the compositions, biomimetic scaffold, scaffold or implant can be based upon many factors, including, but not limited to, the shape and/or size of the bone into which the compositions, biomimetic scaffold, scaffold or implant is to be implanted; the percentage of bone density (i.e., the porousness of the remaining bone); and/or the desired rate and distribution of diffusion of the scaffold or implant into the bone; or a combination of such factors. In some aspects, the shape of the compositions, biomimetic scaffold, scaffold or implant can be constructed to match the shape of the bone or vertebral body and thus allow for a more uniform distribution of the compositions, biomimetic scaffold, implant or ex vivo hematoma or the components present in the composition, scaffold, biomimetic scaffold, implant or ex vivo hematoma. Application of the composition, biomimetic scaffold or implant can occur at the time of surgery or in any other suitable manner.

In some aspects, the shape of the depot implant or scaffold can be that of a sphere or cylinder. In some aspects, the shape of the depot implant or scaffold can be that of a sphere or any other patient specific geometries, forms, or shapes as dictated by clinical exigency. In some aspects, the cylinder shape can be at least 5 mm to about 30 cm (or more) in length. In some aspects, the cylinder shape can be at least 1 mm to about 60 mm (or more) in diameter. In some aspects, the cylinder shape can be straight, and/or curved. In some aspects, the cylinder shape can be a straight rod or a curved rod. The cylinder or rod shape can be any shape with a longitudinal axis that can be longer along one direction than in other directions. The cross-sectional shape of the depot across the longitudinal axis can be any shape. In some aspects, the cross-section shape can be elliptical, circular, trefoil, or any other shape. In some aspects, the depot or scaffold can be either straight or curved in such longitudinal direction. The end surface of the depot or scaffold can be shaped such that it is either flat, rounded or convoluted in shape.

The dimensions of the implant depot or scaffold or ex vivo hematoma can depend on the size of the bone defect and the anatomical site treated. In some aspects, the scaffold can be approximately 20% longer than the actual size of the defect, so that it tightly fits and completely fills the volume of the missing bone. For example, if the size of the bone defect is 3 cm, and it is an adult midshaft femur, the implant depot or scaffold or ex vivo hematoma will likely need to be constructed with dimensions, for example, that are about 3-4 cm in diameter and 3.6 cm in length. In some aspects, the implant depot or scaffold or ex vivo hematoma can resemble the size and shape of a given bone defect. In some aspects, the implant depot or scaffold can be chemotactic.

Also disclosed herein are methods of using any of the compositions described herein and combining said compositions with any of the scaffolds described herein to initiate or enhance bone healing. Also disclosed herein are methods of using any of the compositions and biomimetic scaffolds described herein to reconstruct segmental bone defects. Also disclosed herein are methods of using any of the biomimetic scaffold and compositions described herein to reconstruct segmental bone defects resulting from tumors, trauma, or infection, using ecarin to create a biomimetic scaffold or biomimetic scaffold that initiates the normal fracture healing cascade by delivering catalytic amounts of BMP or one or more bone substitutes that then hyperactivates endogenous growth factors locally.

Also disclosed herein are methods of using any of the compositions and biomimetic scaffolds described herein to treat fractures at risk (e.g., in the osteoporotic, diabetic, elderly, or smokers). Also disclosed herein are methods of using any of the biomimetic scaffolds and compositions described herein to treat fractures at risk using ecarin to initiate the normal fracture healing cascade by delivering catalytic amounts of BMP or one or more bone substitutes that then hyperactivates endogenous growth factors locally.

Also disclosed herein are methods of using any of the compositions and biomimetic scaffolds described herein to treat atypical femur fractures. Also disclosed herein are methods of using any of the compositions and biomimetic scaffolds described herein to treat atypical femur fractures percutaneously using ecarin to create an ex vivo hematoma that initiates the normal fracture healing cascade by delivering catalytic amounts of BMP or one or more bone substitutes that then hyperactivates endogenous growth factors locally.

Also disclosed herein are methods of using any of the compositions and biomimetic scaffolds described herein to treat minimally displaced femoral neck fractures. Also disclosed herein are methods of using any of the compositions and biomimetic scaffolds described herein to treat minimally displaced femoral neck fractures percutaneously using ecarin to create an ex vivo hematoma that initiates the normal fracture healing cascade by delivering catalytic amounts of BMP or one or more bone substitutes that then hyperactivates endogenous growth factors locally.

Also disclosed herein are methods of using any of the biomimetic scaffolds and compositions described herein to treat osteoporotic insufficiency fractures (pelvis, spine). Also disclosed herein are methods of using any of the biomimetic scaffolds and compositions described herein to treat osteoporotic insufficiency fractures (e.g., pelvis, spine) percutaneously using ecarin to create an ex vivo hematoma that initiates the normal fracture healing cascade by delivering catalytic amounts of BMP or one or more bone substitutes that then hyperactivates endogenous growth factors locally.

Also disclosed herein are methods of using any of the compositions described herein to augment spinal fusion procedures in conjunction with scaffolds such as spinal cages (either ceramic, PEEK, or metallic alloys). Also disclosed herein are methods of using any of the compositions described herein to augment spinal fusion procedures in conjunction with scaffolds such as spinal cages (either ceramic, PEEK, or metallic alloys) that allow full immediate weight-bearing, provide more stable fixation, and enhance postoperative recovery, using ecarin to induce local formation of an ex vivo hematoma embedded on the substrate comprising the cage (scaffold), that initiates a bone healing cascade by delivering catalytic amounts of BMP or one or more bone substitutes that then hyperactivates endogenous growth factors locally.

Also disclosed herein are methods of using any of the compositions and biomimetic scaffolds described herein to treat delayed union of long bone fractures (percutaneously or open). Also disclosed herein are methods of using any of the compositions and biomimetic scaffolds described herein to treat delayed union of long bone fractures (percutaneously or open) using ecarin to create an ex vivo hematoma, that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein to treat established non-unions of long bone fractures (percutaneously or open). Also disclosed herein are methods of using any of the compositions and biomimetic scaffolds described herein to treat established non-unions of long bone fractures (percutaneously or open) using ecarin to create an ex vivo hematoma, that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein to improve (e.g. accelerate) healing of long bone fractures. Also disclosed herein are methods of using any of the compositions and biomimetic scaffolds described herein to improve (e.g. accelerate) healing of long bone fractures in selected candidates (such as high-performance athletes) to facilitate more rapid recovery, by using ecarin to create an ex vivo hematoma, that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein to accelerate healing of long bone fractures in selected veterinary candidates (such as thoroughbred racehorses). Also disclosed herein are methods of using any of the compositions and biomimetic scaffolds described herein to accelerate healing of long bone fractures in selected veterinary candidates (such as thoroughbred racehorses) to facilitate more rapid recovery, by using ecarin to create an ex vivo hematoma, that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein to facilitate more rapid and predictable dental and maxilla-facial reconstructions. Also disclosed herein are methods of using any of the compositions and biomimetic scaffolds described herein to facilitate more rapid and predictable dental and maxilla-facial reconstructions by using ecarin to create an ex vivo hematoma that initiates a bone formation cascade by delivering catalytic amounts of BMP or one or more bone substitutes that then hyperactivates endogenous growth factors locally.

Also disclosed herein are methods of using any of the compositions and biomimetic scaffolds described herein to and reverse conditions resulting in spontaneous jaw bone resorption. Also disclosed herein are methods of using any of the compositions described herein to and reverse conditions resulting in spontaneous jaw bone resorption, using ecarin to create an ex vivo hematoma to regenerate bone locally.

Also disclosed herein are methods of using any of the compositions and biomimetic scaffolds described herein to treat and/or reverse conditions resulting in spontaneous osteonecrosis. Also disclosed herein are methods of using any of the compositions and biomimetic scaffolds described herein to and reverse conditions resulting in spontaneous osteonecrosis, using ecarin to create an ex vivo hematoma delivered percutaneously or open (such as Kienböck's disease, avascular necrosis of the femoral head, and osteonecrosis of various other anatomic locations including, but not limited to, the femoral condyles).

Also disclosed herein are methods of using any of the compositions and biomimetic scaffolds described herein to treat and/or reverse conditions resulting in spontaneous avascular necrosis of the femoral head where the femoral head has collapsed. Also disclosed herein are methods of using any of the compositions and biomimetic scaffolds described herein to treat and/or reverse conditions resulting in spontaneous avascular necrosis of the femoral head where the femoral head has collapsed, using ecarin to create an ex vivo hematoma delivered in an open procedure following surgical dislocation of the hip.

Also disclosed herein are methods of using any of the compositions and biomimetic scaffolds described herein to treat osteonecrosis resulting from chemotherapy, alcoholism, smoking, or other exogenous agents. Also disclosed herein are methods of using any of the compositions and biomimetic scaffolds described herein to treat osteonecrosis resulting from chemotherapy, alcoholism, smoking, or other exogenous agents, using ecarin to create an ex vivo hematoma delivered percutaneously or open.

Also disclosed herein are methods of using any of the compositions and biomimetic scaffolds described herein to augment any standard fusion procedures. Also disclosed herein are methods of using any of the compositions and biomimetic scaffolds described herein to augment any standard fusion procedures (e.g., hip, knee, ankle, wrist, elbow, shoulder, subtalar joint, any of the limited fusions in the carpus or midfoot, fusions in any of the smaller joints such as the hallux, pollex, or lesser digits, either toes or fingers), using ecarin to create an ex vivo hematoma, that initiates a bone formation cascade by delivering catalytic amounts of BMP or one or more bone substitutes that then hyperactivates endogenous growth factors locally.

Also disclosed herein are methods of using any of the compositions and biomimetic scaffolds described herein to accelerate healing of scaphoid waist fractures. Also disclosed herein are methods of using any of the compositions and biomimetic scaffolds described herein to accelerate healing of scaphoid waist fractures to facilitate more rapid recovery, by using ecarin to create an ex vivo hematoma that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein to reconstruct complex skeletal defects. Also disclosed herein are methods of using any of the compositions and biomimetic scaffolds described herein to reconstruct complex skeletal defects in the skull, whether resulting from trauma, tumour, or infection, by using ecarin to create an ex vivo hematoma that initiates a bone formation cascade by delivering catalytic amounts of BMP or one or more bone substitutes that then hyperactivates endogenous growth factors locally.

Also disclosed herein are methods of using any of the compositions and biomimetic scaffolds described herein to accelerate healing of sternotomies. Also disclosed herein are methods of using any of the compositions and biomimetic scaffolds described herein to accelerate healing of sternotomies associated with open heart surgery to facilitate more rapid recovery, by using ecarin to create an ex vivo hematoma that initiates a fracture healing cascade by delivering catalytic amounts of BMP or one or more bone substitutes that then hyperactivates endogenous growth factors locally.

Also disclosed herein are methods of using any of the compositions and biomimetic scaffolds described herein in joint arthroplasty components with specially adapted bone ingrowth surfaces augmented with ecarin to induce local formation of an ex vivo hematoma embedded on a structural substrate, that more rapidly initiates a bone healing cascade.

Also disclosed herein are methods of using any of the compositions and biomimetic scaffolds described herein with osseointegration stems and components with specially adapted bone ingrowth surfaces augmented with ecarin to induce local formation of an ex vivo hematoma embedded on a structural substrate, that more rapidly initiates a bone healing cascade.

In some aspects, the one or more bone substitutes is not BMP, rhBMP-2, BMP-2, BMP-7, BMP-4, BMP-6, BMP-9, or BMP-14

As used herein, the term “biomimetic hematoma” can be used to refer to an “ex vivo hematoma”.

Also disclosed herein are methods of using any of the compositions described herein to reduce bleeding or manage bleeding.

Also disclosed herein are methods of using any of the compositions described herein to manage widespread venous bleeding/ooze during surgical procedures. In some aspects, in the methods disclosed herein any of the compositions described herein can be formulated to be sprayed on topically as an aqueous aerosol (using an atomizer for ecarin distribution to affected areas).

Also disclosed herein are methods of using any of the compositions described herein to manage bleeding or stop bleeding from individual injured vessels (e.g., massive bleeder) intra-operatively or in an emergency casualty situation. In some aspects, in the methods disclosed herein any of the compositions described herein can be administered on a bead (e.g. magnetic bead). In some aspects, in the methods disclosed herein any of the compositions described herein can be applied as a clamp/clamshell on the end of a vessel to simultaneously clamp off and deliver ecarin locally, restricting the application to the specific injured vessel end. The clamp or clamp element can constrict the adjacent injured vessel adjacent, and can eliminate or minimize the risk of systemic administration of the compositions.

Also disclosed herein are methods of using any of the compositions described herein as a selective embolization. In some aspects, in the methods disclosed herein any of the compositions described herein can be delivered via an interventional radiologist to one or more targeted blood vessels to manage or stop intra-pelvic/intra-abdominal/oesophageal/intra-cranial bleeding using a long radiographically directed catheter that then allows selective and highly specific administration of ecarin limited to discrete pathology as indicated (e.g., similar to methods carried out using angiographic coils).

Also disclosed herein are methods of using any of the compositions described herein to treat menometrorrhagia. In some aspects, the methods disclosed herein can be used to direct the installation or placement of any of the compositions described herein into the uterus in affected women. In some aspects, ecarin can be formulated to be delivered as part of a bio-degradable collagen bead(s).

Also disclosed herein are methods of using any of the compositions described herein to treat hemophiliac associated spontaneous hemarthrosis. In some aspects, ecarin can be formulated to be delivered as part of a bio-degradable collagen bead(s).

Also disclosed herein are methods of using any of the compositions described herein to treat spontaneous hemarthrosis related to an anti-coagulant overdose (e.g., warfarin, coumadin, etc.). In some aspects, ecarin can be formulated to be delivered as part of a bio-degradable collagen bead(s).

Also disclosed herein are methods of using any of the compositions described herein to treat spontaneous intra-muscular bleeds related to an anti-coagulant overdose (e.g., warfarin, coumadin, etc.). In some aspects, in the methods disclosed herein any of the compositions described herein can be used as a selective embolization.

Also disclosed herein are methods of using any of the compositions described herein to treat spontaneous intra-muscular bleeds related to haemophilia. In some aspects, in the methods disclosed herein any of the compositions described herein can be used as a selective embolization.

Also disclosed herein are methods of using any of the compositions described herein to treat post-operative hemarthrosis in any elective total knee replacement. In some aspects, ecarin can be formulated to be delivered as part of a bio-degradable collagen bead(s) or nanoparticle(s). In some aspects, the bio-degradable collagen bead(s) or nanoparticle(s) can be delivered or sprinkled liberally into the joint immediately prior to closure of the wound.

Also disclosed herein are methods of using any of the compositions described herein to treat epistaxis. In some aspects, ecarin can be formulated to be delivered as part of a bio-degradable collagen bead(s). In some aspects, the bio-degradable collagen bead(s) can be embedded in a fabric packing material or enclosed within a fabric sheath to limit their distribution and contain them locally. In some aspects, ecarin can be delivered in the form of a nasal pack, such that the ecarin is formulated to be a part of a bio-degradable collagen bead(s) that is embedded in a fabric packing material or enclosed within a fabric sheath.

Also disclosed herein are methods of using any of the compositions described herein to treat retinal haemorrhage. In some aspects, in the methods disclosed herein any of the compositions described herein can be used as a selective embolization. In some aspects, ecarin can be formulated to be delivered as part of a bio-degradable collagen bead(s) or nanoparticle(s). In some aspects, the bio-degradable collagen bead or nanoparticle formulation can be used to create a Velcro-type effect by creating a self-adherent geometry to minimize the risk of recurrence and actively address retinal detachment.

In some aspects, “bleeding” can be a hemorrhage. In some aspects, blood can be escaping the circulatory system from one or more damaged blood vessels. In some aspects, bleeding can be internal or external.

### Articles of Manufacture

The compositions and biomimetic scaffolds described herein can be packaged in a suitable container labeled, for example, for use as a therapy to treat bone defects or any of the methods disclosed herein. In some aspects, the composition comprising the ex vivo hematomas described herein can be packaged in a suitable container labeled, for example, for use as a therapy to treat bone defects or any of the methods disclosed herein, and can be packaged separately from the scaffold portion of the biomimetic scaffold. Accordingly, packaged products (e.g., scaffolds, sterile containers containing the compositions including the individual components of any of the compositions or ex vivo hematomas described herein and packaged for storage, shipment, or sale at concentrated or ready-to-use concentrations) and kits, including at least isolated whole blood and sodium citrate; or platelet rich plasma, plasma, or plasma with red blood cells; and ecarin; oscutarin and calcium chloride; calcium chloride; thrombin; or thrombin and calcium chloride as described herein and instructions for use, are also within the scope of the disclosure. A product can include a container (e.g., a vial, jar, bottle, bag, or the like) containing the biomimetic scaffold or composition or ex vivo hematomas described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape). The legend can be associated with the container (e.g., affixed to the container) and can describe the manner in which the biomimetic scaffold, compositions or ex vivo hematomas therein should be administered (e.g., the frequency and route of administration), indications therefore, and other uses. The biomimetic scaffolds or compositions or ex vivo hematomas can be ready for administration (e.g., present in dose-appropriate units), and may include a pharmaceutically acceptable adjuvant, carrier or other diluent. Alternatively, the compounds can be provided in a concentrated form with a diluent, with accompanying instructions for dilution.

### EXAMPLES

### Example 1: Absorbable Collagen Scaffold in Combination with Coagulated Whole Blood and rhBMP-2 Successfully Repairs Large Bone Defects in Rats

One of the most promising alternatives to autologous bone grafting is the use of recombinant human bone morphogenetic protein 2 (rhBMP-2) delivered on an absorbable collagen sponge (ACS). However, successful bone healing requires a supra-physiological dose of rhBMP-2 due to its burst release and short half-life in vivo, most of which rapidly leaches away as soon as the collagen sponge becomes compressed after insertion into the bone defect area. This results in an increased incidence and severity of ectopic/heterotopic ossification and many other related side effects, such as antibody formation, implant dislodgment, bone resorption, and even cancer. A more suitable carrier is clearly required to treat these complex bone injuries and to improve the effectiveness of BMPs by significantly reducing the dose required, thus mitigating their potentially dangerous side effects.

The hematoma that forms at the site of the fracture serves as an endogenous scaffold that activates a cascade of biological events incorporating molecular factors derived from the surrounding tissues. Studies have shown that the hematoma formed at the fracture site significantly influences the way fractures heal, and that removal of this hematoma delays fracture healing. To increase the effectiveness of the ACS, it was combined with an ex vivo hematoma that mimics the intrinsic structural and biological properties of the endogenous fracture hematoma. It was tested whether an ex vivo hematoma that is created by combining a mixture of whole blood, coagulants, and a low dose of rhBMP-2 to the ACS, would significantly enhance the regenerative capacity of bone and reduce the dose of rhBMP-2 required compared to using an ACS and BMP-2 alone in a rat femoral 5 mm large critical size defect model.

Materials and Methods. Preparation of the ACS/whole blood biomimetic scaffold. Blood was drawn from anesthetized male donor Fisher 344 rats by terminal bleeding at the time of euthanasia. The blood was collected by cardiac puncture using a 21-gauge needle. Nine parts of blood were mixed with one part of 3.2% trisodium citrate solution to prevent coagulation. To create a biomimetic scaffold with a cylindrical shape, 0.55 μg of rhBMP-2, 10 mM of calcium chloride, and 5 U/mL of thrombin (ex vivo hematoma) were pipetted on the bottom of flat-bottom wells of a 96-well-plate before adding citrated blood. The solution was mixed by pipetting up and down and was quickly transferred to another well that contained a cylindrical dry ACS. The biomimetic scaffold was incubated at room temperature for 15 minutes before being implanted into a 5 mm femoral defect in a rat. ACS without whole blood was used as a control group.

Results. The bone defects treated with ACS/WB/BMP2 were fully bridged 4 weeks after surgery. At 8 weeks, the bone defect healing was complete, with the cortical bone almost fully restored to its original shape with a small amount of intramedullar trabecular bone remaining, indicating advanced healing and remodeling. In contrast, bone defects treated with ACS/BMP2 failed to heal within 8 weeks, consistently resulting in non-unions (FIG. 1).

Conclusion. The data show that by adding a mixture of whole blood and coagulants at specific concentrations (mimicking natural fracture hematoma, ex vivo hematoma) to the ACS, the biomimetic scaffold was able to deliver rhBMP-2 with a much higher efficiency compared to the ACS alone. Moreover, this healing was achieved using a significantly lower dose of rhBMP-2, while the same dose delivered on ACS was unable to even initiate the healing of the bone defects. The superior healing is likely due to the biometic scaffold more closely resembling an innate fracture hematoma which is a endogenous reservoir for important growth factors, such as VEGF, FGF, TGF, and therefore, binds the rhBMP-2 more efficiently than the ACS alone. Furthermore, the added benefit of adding the ex vivo hematoma to the ACS is that the delivered rhBMP-2 is safely contained within the scaffold, which can prevent any ectopic/heterotopic ossification or other deleterious side effects. The use of this biomimetic scaffold enables the delivery of rhBMP-2 at significantly reduced doses without compromising the healing outcome.

### Example 2: Ex Vivo Hematoma in Combination with Various Bone Substitutes

Ex vivo hematomas can be mixed with any of the bone substitutes derived from biological products currently available on the market such as DBX, DBM, corals, allogeneic cancellous bone chips, or bone marrow aspirate concentrate (BMAC), including bone graft from long bones harvested using the reamer irrigation aspirator (RIA). Also, ex vivo hematomas can be mixed with synthetic bone substitutes such as calcium sulfate, calcium phosphate cements, beta-tri-calcium (TCP) phosphate ceramics, biphasic calcium phosphates (Hydroxyapatite (HA) and Beta-TCP ceramics), bioactive glasses, polymer-based bone substitutes.

Demineralized bone matrix (DBM) is a cancellous and cortical bone substitute that is available as a dry, moldable or injectable form, and can exist as a paste, powder, putty, granules, gel, sponge, or strips. DBM is prepared from allograft bone by decalcification, and provides a framework for cell and matrix protein adhesion, and it also contains osteogenic substances that induce new bone growth. The osteogenic components of DBM are a mixture of growth factors, including many from the transforming growth factor-beta family (TGF-B) such as BMP-2. DBM provides little biomechanical strength, but functions as an osteoconductive, and to a lesser degree, as an osteoinductive material. In orthopedics, DBM is used as an autologous bone graft extender, and although they are good when used as additives (extenders) in cases where limited autologous graft is available, as a stand-alone product they are unable to initiate bone formation. This is especially true of large bone defects due to processing, donor selection, terminal sterilization, and additives (such as hyaluronate, plurionic acid, glycerine, or gelatin), which yield products with variable biological activities, and consequently, unpredictable biological osteoinductivity and clinical performance. The same applies for many of the other bone substitutes currently available on the market.

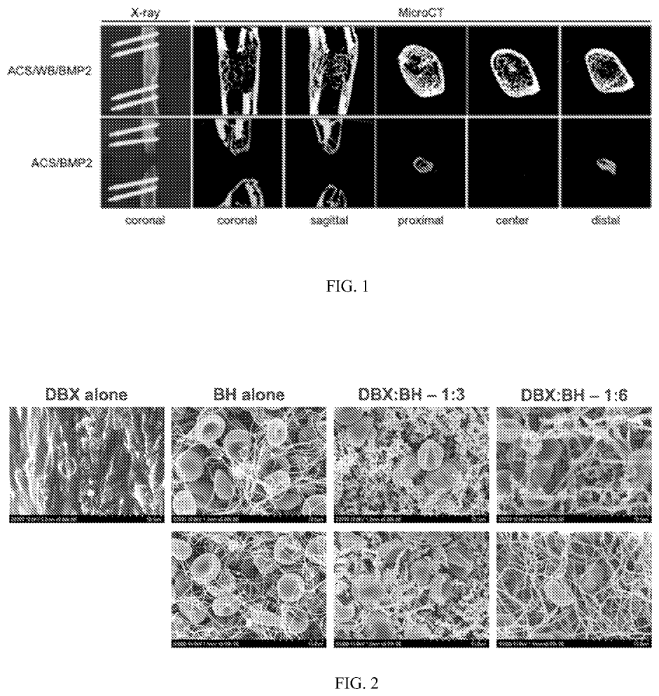

By contrast, the ex vivo hematoma was developed to mimic healing fracture hematomas and successfully and efficiently deliver growth factors, such as rhBMP-2, to large segmental bone defects that do not heal spontaneously on their own. The main concept was to use autologous blood in combination with specific concentrations of coagulants in order to mimic an endogenous fracture hematoma, because a hematoma formed at the time of injury is the body's contains specific micro-architectural properties that activate the cascade of biological events which in turn initiates bone healing. This blood clot (hematoma) typically contains the components required to initiate the bone healing cascade, including blood cells such as erythrocytes, leukocytes, and platelets. In fact, platelets play an important role in bone healing by releasing and activating important angiogenic and osteogenic growth factors. Experimental results using the ex vivo hematoma as a delivery vehicle were successfully healed critical sized defects in rats with using a fraction of the BMP-2 dose necessary for healing in this model. In contrast, absorbable collagen sponges were completely unable to initiate the healing process using such a small dose. Moreover, the bone quality was significantly better than when BMP-2 was delivered on an absorbable collagen sponge. To build on these results, the ex vivo hematoma in combination with one or more bone substitutes, such as DBM, was tested to see if such combination would enhance the hematoma's efficacy, and therefore be more efficient and robust in bone defect regeneration and healing. For example, FIG. 2 demonstrates the architecture of one of the demineralized bone matrix products (DBX), and it clearly illustrates that a solid material does not have the porosity that is important for cell recruitment and infiltration. However, once DBM is mixed with the ex vivo hematoma, the resulting scaffold has a microarchitecture that mimics the endogenous healing fracture hematomas. Moreover, as a combination product, the ex vivo hematoma comprise the important ingredients to promote the healing of fractures. Furthermore, adding DBM increases the amount of growth factors available at the site of the bone defect to efficiently and robustly initiate bone regeneration. Therefore, a structurally well-organized ex vivo hematoma comprising DBM can also serve as a temporary reservoir for the continuous release of growth factors, and it can provide a suitable environment that encourages cell infiltration, proliferation, and differentiation from the surrounding tissues.

### Example 3: Ex Vivo Hematoma Combined with a Scaffold

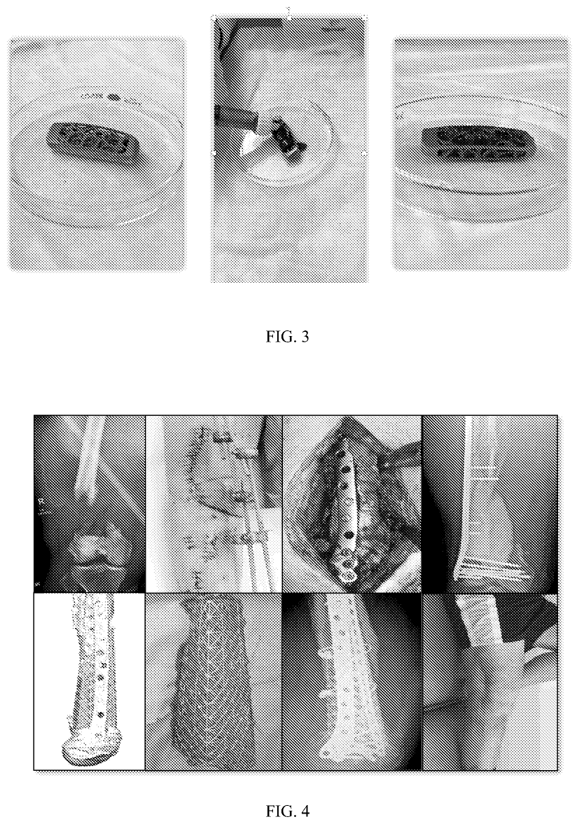

Patient-specific 3D printed titanium cages have been employed for the treatment of massive segmental bone loss and spinal fusions (FIGS. 3 and 4). These titanium cages are used to address segmental bone defects exceeding 6 cm in length as well as for spinal pathologies. These metal implants or scaffolds are not osteoinductive, and therefore, are unable to initiate bone regeneration and incorporation within the pores of these titanium scaffolds. To initiate bone healing and regeneration, orthopaedic surgeons currently augment their regenerative potential by adding autologous bone grafts, bone substitutes, and growth factors; however, none of these products are able to initiate reliable, consistent, and robust bone regeneration, especially in long bones. Furthermore, these specific options are unfortunately associated with many disadvantages. For example, autologous bone grafts are often not readily available at the volumes required, and there are issues of potential donor site morbidity. Additionally, these grafts are not user friendly because they have to be manually pressed into the voids/pores of the cage/scaffold by a surgeon (FIG. 4), which is time consuming, and almost impossible to achieve uniform graft incorporation within the titanium cage/scaffold, for instance at the center of the cage. Growth factors also have to be delivered in high concentrations, which has been shown to be associated with many deleterious side effects such as ectopic bone formation. Moreover, uniform incorporation within the cage/scaffold is not a straightforward procedure, often requiring the growth factors to be used in combination with bone substitutes. Of course bone substitutes themselves are also associated with a variety of disadvantages that are very similar to those of autologous bone grafts and growth factors, with the main issues being unpredictable biological osteoinductivity and clinical performance, along with having to be manually pressed into the voids/pores of the cage/scaffolds.

To overcome this problem, the ex vivo hematoma, whether in combination with growth factors or bone substitutes such as DBM, facilitates more uniform incorporation within the titanium cage/scaffold, because the ex vivo hematoma starts off as a liquid (FIG. 3) and penetrates completely, while also enhancing consistent bone regeneration. This treatment strategy works successfully because the bone substitutes are delivered within the ex vivo hematoma itself, which mimics the micro-architectural properties of an endogenous healing fracture hematoma. This results in the increased amount of growth factors required to efficiently and robustly initiate bone regeneration. Moreover, a structurally well-organized scaffold (e.g., the ex vivo hematoma) will also serve as a temporary reservoir for the continuous release of growth factors, and it provides a very favorable environment that encourages cell infiltration, proliferation, and differentiation from the surrounding tissues.

### Example 4: Percutaneous Delivery of the Ex Vivo Hematoma for the Treatment of Delayed Union, Nonunion, and Bone Defects

A mixture of the ex vivo hematoma with either bone substitutes (e.g., DBM, bone chips) and/or growth factors (e.g., BMP-2, PDGF) can be delivered percutaneously to enhance the healing of delayed bone union, nonunions, and bone defects.

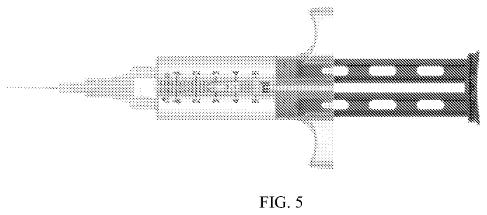

The method is as follows: Similar to an arthroscopic or endoscopic procedure, a surgeon inserts a narrow tube (plastic or metallic) as a cannula to allow the introduction of a fiber-optic video camera through a small incision, roughly the size of a buttonhole. The view inside the delayed/nonunion or bone defect can be transmitted to a high-definition video monitor. Another small incision is made to introduce a second small tube (cannula), through which a rotating blade is inserted to gradually deploy like an umbrella as it enters into the nonunion site, and this position can be monitored fluoroscopically. Expanding about 5-10 mm from the center of the nonunion, to allow for limited debridement to clear fibrotic tissue from within the nonunion site and prepare space for the injection of the ex vivo hematoma comprising a scaffold. This step creates a void to deliver biologics as well as remove dense, avascular fibrotic tissue which is known to interfere with successful bone healing. An optional third small incision can be created to insert another cannula/tube to remove and irrigate the resulting debris from debridement of the fibrotic nonunion tissue. Once the nonunion site is prepared for the delivery of the ex vivo hematoma, a syringe with two chambers, for example, can be used (FIG. 5). Through the same tube that was used to remove the nonunion fibrotic tissue, a long needle can be inserted attached to the two-chamber syringe. One of the chambers will contain the coagulants (calcium and thrombin, or ecarin), and the second chamber will contain either whole blood alone or in combination with exogenous growth factors (e.g., BMP2, PDGF, VEGF) and/or bone substitutes (e.g., DBM, allogeneic cancellous bone chips). During the application, upon mixing as it enters the needle, the interaction of these components form the ex vivo hematoma to initiate the bone healing process. After the ex vivo hematoma is delivered to the nonunion site, the cannulas are removed, and incisions are closed with sutures. This procedure can result in less pain for patients, limit the risk of infection and other complications, minimize surgery time, and reduce the time it takes the patient to recover and return to normal activities.

### Example 5: Structural and Biological Properties of Hematomas

An approach to healing large segmental bone defects. It has been well established that hematoma formed at the fracture site significantly influences the way fractures heal. For example, studies have shown that the removal of a hematoma delays fracture healing (Schell H, Peters A, Duda G N. Removal of fracture hematoma and replacement with fresh hematoma delays bone healing in sheep. Bone. 2012). Furthermore, there are reports that suggest the structural properties of a formed fibrin clot, such as porosity and thickness of fibrin fibers, influences bone repair (Wang X, Friis T E, Masci P P, Crawford R W, Liao W, Xiao Y. Alteration of blood clot structures by interleukin-1 beta in association with bone defects healing. Sci Rep. Nature Publishing Group; 2016; 6: 35645; and Wang X, Friis T, Glatt V, Crawford R, Xiao Y. J Tissue Eng Regen Med. 2017; 11: 2864-2875.) The results described herein demonstrate that in vivo hematomas isolated from 0.5 mm rat femoral defects (normally healing fractures) had 35% thinner fibrin fibers (209±20 nm) as compared to 5 mm segmental bone defects (do not heal without an intervention; 320±64 nm) 3 days after surgery. Furthermore, a less porous network was also observed in 5 mm defects compared to 0.5 mm (42.56% vs 50.03%), resulting in a 16% difference between the groups. To investigate whether there are differences in biological properties between the hematomas formed in large bone defects (5 mm) and normally healing fractures (0.5 mm), an in vivo study was performed using RNA sequencing analysis.

Major differences were found for genes mediating the inflammatory response (e.g. Il1b—produced by activated macrophages; Sdf1—expressed in the area of inflammatory bone destruction, where it mediates their suppressive effect on osteoclastogenesis), which were predominantly upregulated in 5 mm vs. 0.5 mm defects. Genes important for extracellular matrix (ECM) structural constituents (e.g. Col1a1, Col2a1, Col3a1), as well as ECM proteases (e.g. Mmp2) and their inhibitors (e.g. Timp1) were mostly downregulated. In contrast, genes modulating cell adhesion molecules (e.g. Thbs1) were upregulated. The analysis of genes involved in the processes of angiogenesis showed that angiogenin (Ang), a potent stimulator of new blood vessel formation, was downregulated, while an angiogenic cytokine, endothelin (Edn1), a potent vasoconstrictor that also stimulates cells in the osteoblast lineage, was upregulated. The expression of genes related to osteogenesis demonstrated a significant portion of downregulated genes involved in osteoblast differentiation (e.g. Bglap, also known as osteocalcin—secreted by osteoblasts to initiate bone repair) and bone formation (e.g. Bmp7—plays a key role in the transformation of mesenchymal cells into bone and cartilage).

These results are the first to demonstrate important differences in gene expression between normally healing fractures and large bone defects during the earliest phases of bone healing. The most striking differences were found in the set of genes involved in the inflammatory response, a key event after fracture. The upregulation of inflammatory genes in 5 mm vs. 0.5 mm defects suggests that a large bone defect induces a stronger inflammatory response than does a normal fracture, which leads to an increased recruitment of macrophages, fibroblasts, MSCs, and osteoprogenitor cells. The invading inflammatory cells also produce proangiogenic cytokines, which explains the upregulation observed when comparing 5 mm vs. 0.5 mm defects. At the same time, the downregulation of many genes important for skeletal development, bone mineral metabolism, and the formation of an ECM, suggests there is a reduced osteogenic response in large bone defects.

An in vitro study was also conducted to determine whether an ex vivo blood clot could be produced with specific structural properties using a SVCE, ecarin, and to determine whether ecarin is toxic to stem cells. The results convincingly showed that the structural properties of blood clots varied depending on the concentration of ecarin. For instance, higher concentrations of ecarin resulted in thinner fibrin fibers with an average thickness of 93±3 nm, whereas at the lowest concentration, the thickness of fibrin fibers was 173±9 nm. The rate of cell proliferation from 1 to 7 days was lower with an increasing concentration of ecarin. For example, there was a 4.3±0.7-fold increase at the highest concentration, whereas at the lowest concentration there was a 13.7±3.1-fold increase, compared to 14.8±2.6-fold increase without ecarin. Cells cultured within ex vivo hematomas had stable cell numbers over a 7-day period. Thus, these results suggest that ecarin does not cause toxicity, however, higher concentrations might result in lower cell proliferation rates.

Based on these observations, additional experiments will be carried out to determine the structural and biological properties of formed hematomas in normal fractures, which heal, and compare them to large bone defects, which do not heal without intervention (see Examples below). For example a well-organized fibrin clot will be studied for its ability to enhance bone healing by serving as a temporary reservoir for the continuous release of growth factors, and by providing adequate space to assist cell infiltration, proliferation, and differentiation. Therefore, the main opportunity for enhancing the repair process lies with the production of a blood clot that mimics intrinsic properties of healing fracture hematoma. The structural properties of an ex vivo produced hematoma can be altered using SVCEs, thereby mimicking intrinsic fracture hematoma which, when implanted into a segmental bone defect, will enhance and accelerate bone healing. Various snake venoms have been used successfully in patients with heart disease, cancer, and strokes as well as for diagnostic purposes for diseases such as lupus. Thus, these experiments will also be the first to examine the use of the snake venom enzyme, ecarin, as an alternative clotting agent compared to the addition of bovine thrombin, which has been shown to have side effects when used clinically (Diesen D L, Lawson J H. Vascular. 2008; 16: S29-36; Ofosu F A, Crean S, Reynolds M W. Clin Ther. 2009; 31: 679-691; and Sands J J, Nudo S A, Ashford R G, Moore K D, Ortel T L. Am J Kidney Dis. 2000; 35: 796-801). This approach will be explored as an improved treatment strategy, as it is a bio-scaffold that can be used to enhance bone healing more reliably by either completely eliminating the need of growth factors such as BMPs, or significantly minimizing the doses required to enhance the repair process of bones. The result is a more innate treatment strategy than what is currently available; it would offer significant cost savings and, most importantly, eliminate the many adverse side effects associated with high doses of BMPs. Furthermore, the results described herein may have significant implications for the treatment of bone injuries to the general public as well as to the service personnel.

### Example 6: Structural and Biological Properties of In Vivo Fracture Hematomas

Bone defects were created in groups of male SAS Fischer rats (10-12 weeks of age, n=38; n=5-8/group), and stabilized with external fixators (RISystem AG; Glatt V, Evans C H, Matthys R. Eur Cell Mater. 2012; 23: 289-98; discussion 299; and Glatt V, Miller M, Ivkovic A, Liu F, Parry N, Griffin D, et al. J Bone Joint Surg Am. 2012 Nov. 21; 94(22):2063-73) to characterize and compare the structural and biological properties of hematomas formed during normal bone healing (0.5 mm) and large segmental bone defects (5 mm). To evaluate the course of fracture healing, a 0.5 mm osteotomy, which is also possible with the same external fixation device, was performed. The reason for using osteotomies is that they are reproducible and allow for a more consistent size of hematoma formation, which is important for characterization of hematoma structural properties. Animals were sacrificed at 3 days following surgery to evaluate the structural properties of the fracture hematoma, after the blood clots are mature. Structural properties of hematomas, such as thickness, density, and porosity of the fibrin fibers were assessed using scanning electron microscopy (SEM; n=8/group) and ImageJ software. Furthermore, a different set of samples were used to analyze differentially expressed genes involved in the initiation of the bone repair process using RNA sequencing (n=6/group). Histology and immunohistochemistry (IHC) will also be performed (n=5/group) to characterize the tissues and confirm the presence of key proteins involved in the initiation of the repair process, such as macrophages (CD68, CD40, and CD206) osterix, PECAM1, vWF, VEGF, type I collagen, and H&E for an overall tissue morphology. The results were used to determine whether there is a correlation between the structural properties and the expression of specific genes and proteins in 3 day-old hematomas.

Methods. Rat, Large Critical Sized Defect and Osteotomy Models. Male SAS Fischer rats (Charles River Laboratories, Inc., Wilmington, MA, USA), weighing approximately 200-250 g (10-12 weeks old) were anesthetized by the administration of isoflurane (2%; 21/min) with a small animal vaporizer. The animals then received intramuscular injections of 20 mg/kg of cefazolin (antibiotic) and 0.08 mg/kg buprenorphine (analgesic) into the left thigh. Detailed information of the surgical procedure can be found (Glatt V, Matthys R. Adjustable stiffness, external fixator for the rat femur osteotomy and segmental bone defect models. J Vis Exp. 2014). Briefly, the right hind leg of each animal was shaved, disinfected with chlorhexidine, placed in a sterile field and covered with a sterile surgical drape exposing only the right leg. An incision of approximately 3.5-4 cm was made through the skin running anterior-lateral on the surface of the right femur from the greater trochanter to the supracondylar region of the knee. The shaft of the femur was exposed by gentle dissection between the quadriceps and hamstring muscles. The external fixator bar was first clipped on the Gigli wire saw guide, and then placed on the anterior-lateral aspect of the femur to guide the drill and permit reproducible positioning of four drill holes using a pen drill (RISystem AG, Davos Platz, CH). The mounting pins were inserted into the predrilled holes one at a time, starting at the proximal side. After the fixator was in place, the saw guide was used to make the defects. For this, a Gigli wire saw was passed through the two grooves underneath the femur to create a 5 mm segmental defect by reciprocal motion back and forth, and a single wire saw was used to create a 0.5 mm defect. When the defects have been created, the saw guide was removed, and the wound was closed in layers. The rats were given analgesic every 12 hours and antibiotic every 24 hours for three days post-surgery. Hematomas were collected at 3 days for structural and biological analyses.

Scanning Electron Microscopy. The in vivo fracture hematomas and ex vivo blood clots were treated similarly. The samples were fixed in 4% paraformaldehyde overnight. Gross morphology was captured at 100-1,000× magnification using the Hitachi SU1510 VP-SEM. For analyses of fiber diameter and density, the samples were post-fixed in 4% osmium tetroxide and dehydrated through a gradient of ethanol solutions (25-100%). Slices of the hematomas and blood clots were then dried using the Leica EM Critical Point Dryer, mounted on silicon chip specimen supports, and sputter-coated with gold-palladium before being imaged at 10,000× (Hitachi S5500 SEM/STEM) to reveal structural properties at high resolution. The images were analyzed using ImageJ.

RNA-Sequencing. Parallel sequencing of RNA (RNA-Seq) is a high throughput method that allows global measurement of gene transcript abundance (Wang Z, Gerstein M, Snyder M. RNA-Seq: A revolutionary tool for transcriptomics. 2009. pp. 57-63). To determine differential expression of genes in the fracture hematomas, samples were collected into microcentrifuge tubes, immediately snap-frozen in liquid nitrogen and stored at −80° C. RNA extractions were performed using the QIAGEN RNeasy Plus Universal Tissue Mini (QIAGEN, Inc., Germantown, MD, USA) following the manufacturer's protocol. The concentration and quality of the RNA were determined using a nanodrop spectrophotometer (ND-1000, Thermo Fisher Scientific, Inc.), and RNA integrity was assessed using an Agilent 2100 Bioanalyzer (Agilent Technologies, Inc., Santa Clara, CA, USA) according to the manufacturer's protocol. The bone cylinder, which is removed to create 5 mm bone defects, was used as a control representing the gene expression status of healthy bone. The global transcriptome analysis was used to identify up-regulated and/or down-regulated genes that have a major influence in the initiation process of bone repair. This work was performed using the Illumina HiSeq 3000.

Histology and Immunohistochemistry. Samples will be stained with hematoxylin and eosin to view gross tissue morphology. Standard immunohistochemistry protocols will be applied to paraffin embedded sections (5 μm). The sections will be labeled with a panel of antibodies to determine the spatial expression of proteins such as macrophages (CD68 and CD206), osterix, PECAM1, type II collagen, type X collagen (Maes C, Kobayashi T, Selig M K, Torrekens S, Roth S I, Mackem S, et al. Osteoblast precursors, but not mature osteoblasts, move into developing and fractured bones along with invading blood vessels. Dev Cell. Elsevier Ltd; 2010; 19: 329-344).

To carry out these experiments, a 5 and 0.5 mm femoral bone defect model was used, and stabilized with the external fixation device described. This is a well-established research model. The major outcome measures that were used are scanning electron microscopy, RNA sequencing, and histology/immunohistochemistry, which are routine procedures. As disclosed herein, data using the SEM and RNA sequencing analysis showed that there are discernible differences in structural and biological properties between hematomas formed in the 0.5 mm defect compared to the 5 mm defect.

Scanning electron microscopy images depict the structural properties of fracture hematomas. For example, clear structural differences between normally healing fractures (0.5 mm) and large bone defects (5 mm) can be observed. More specifically, the normal fracture (0.5 mm) showed a more porous, less dense and thinner fibrin fibers, and rougher surface, while the large bone defect (5 mm) showed a less porous, denser and thicker fibrin fibers, and smooth surface.

### Example 7: Structural Properties of an Ex Vivo Formed Blood Clot/Hematoma Using a Snake Venom Clotting Enzyme

The SVCE, ecarin, was used to alter the structural properties of blood clots in an effort to mimic the properties of natural hematomas formed in a 0.5 mm osteotomy model using various concentrations of ecarin. Whole blood was collected from the same animals used in Example 4 by cardiac puncture using a 21-gauge needle at the time of euthanasia. The anticipated yield of blood was approximately 5-10 mL per animal. The blood was mixed with one part 4% sodium citrate solution to nine parts of blood to prevent coagulation. The purified enzyme from Saw-scaled viper venom, ecarin, was purchased from Sigma (Sigma-Aldrich Co., St. Louis, MO, USA). Variables such as pH, ionic strength and calcium was kept constant, whereas the prothrombotic enzyme was applied across a range from pico to nano molar for the final concentrations. Calcium chloride (CaCl₂) and CaCl₂+recombinant human thrombin was used as controls. The ex vivo blood clots are intended to have a cylindrical shape with a height of 5 mm to span the defect size, and a diameter of 4 mm, consistent with the average diameter of a rat femur. Blood clots with a height of 0.5 mm were used as controls to determine if the same concentration of SVCE added to a smaller volume of blood produces the same structural properties in the formed blood clot. Structural changes, in particular the thickness and density of fibrin fibers, and the overall clot structure, was evaluated using SEM. Using the methods described herein blood clots in rats can be observed with different structural properties.

Preparation of Ex Vivo Blood Clots. To create ex vivo blood clots, 5-10 mL whole blood was collected from anesthetized rats by cardiac puncture at the time of euthanasia. Immediately after collection, the blood was mixed 9:1 with 4% sodium citrate solution to prevent coagulation. A range of different concentrations of ecarin was used to induce blood clotting. The samples were left at room temperature for 2 hours to allow full coagulation. Subsequently, the ex vivo blood clots were fixed in 4% PFA at 4° C. overnight, before being processed for scanning electron microscopy.

Scanning Electron Microscopy. The in vivo fracture hematomas and ex vivo blood clots were essentially treated in the same manner. The samples were fixed in 4% paraformaldehyde overnight. Gross morphology was captured at 100-1,000× magnification using the Hitachi SU1510 VP-SEM. For analyses of fiber diameter and density, the samples were post-fixed in 4% osmium tetroxide and dehydrated through a gradient of ethanol solutions (25-100%). Slices of the hematomas and blood clots were then be dried using the Leica EM Critical Point Dryer, mounted on silicon chip specimen supports, and sputter-coated with gold-palladium before being imaged at 10,000× (Hitachi S5500 SEM/STEM) to reveal structural properties at high resolution. The images were analyzed using ImageJ. As disclosed herein the SVCE, various concentrations of ecarin were used to alter the structural properties of blood clots (ex vivo hematoma), using either whole blood or platelet rich plasma (PRP) in an effort to mimic the properties of hematomas formed in a 0.5 mm osteotomy model. Ecarin (0.1 and 0.5 U/mL) or CaCl₂ (10 mM) was used as coagulants. Results disclosed herein demonstrated that it is possible to create ex vivo hematoma with the required structural properties by varying concentrations of ecarin, using either whole blood or PRP.

Scanning electron microscopy images show the structural properties of ex vivo snake venom-induced ex vivo hematomas. These results show that the morphology of the blood clots can be manipulated using a snake venom enzyme or calcium chloride which affects fibrin fiber thickness and density, and that the structural properties of the ex vivo hematomas were different from those that were created with whole blood compared to those created with PRP.

### Example 8: To Determine if this Ex Vivo Hematoma Provides a Viable Environment for Mesenchymal Stem Cells (MSCs)

These experiments will be performed to determine the ability of MSCs to survive in the presence of ecarin as well as when seeded within ecarin-induced blood clots to assess the biocompatibility of these scaffolds. Cell viability and the potential cytotoxicity of the ecarin at various concentrations will be measured in 2D and 3D assays. Rat bone MSCs will be cultured in the presence of different concentrations of ecarin to determine cell viability. Cell proliferation (e.g., CyQUANT™ Cell Proliferation Assay Kit) and cytotoxicity (e.g., Vybrant™ Cytotoxicity Assay Kit) will be measured at days 1, 7, 14, and 21. Subsequently, ecarin will be added to a mix of citrated blood at a concentration established from the previous experiments, taking into account cell survival along with the specific structural properties determined. After coagulation, the blood clots will be transferred into 24-well-plates containing growth medium.

To test the biocompatibility of the ecarin-induced blood clots, samples will be removed from the culture on days 1, 7, 14 and 21 (n=3/group) to study the survival rate and differentiation potential of the seeded cells, as well as their ability to form an extracellular matrix. Furthermore, the cell numbers required in the blood clots to maximize the regenerative capacity of bone tissue will be determined. Cell viability within the blood clots will be evaluated using the LIVE/DEAD® cell viability assay and confocal imaging.

To test the cells' ability to differentiate, the cells will be cultured in adipogenic, chondrogenic, and osteogenic differentiation media. qRT-PCR will be used to determine the differential expression of genes at the selected time points. Once cell viability is established, the hematoma that most resembles the structural and biological properties of natural hematomas during 0.5 mm defect/osteotomy bone healing will be implanted in an in vivo rat, 5 mm femoral defect model to study their ability to heal large segmental bone defects. Based upon results in FIGS. 5A, B, it is expected that using lower concentrations of coagulating enzyme (<0.5 U/mL), ecarin, will not be toxic to the cells.

Cell culture. Rat bone marrow stem cells will be cultured according to standard protocols. Medium will be replaced every 3-4 days.

2D Cell Viability Assay. PrestoBlue™ Cell Viability Reagent (Thermo Fisher Scientific, Inc., Waltham, MA, USA) will be added directly to the culture medium of rat bone marrow stem cells grown in well-plates at 1, 3, and 7 days. After a 20 min incubation at 37° C., fluorescence will be read in a multiplate reader according to the manufacturer's instructions.

2D Cell Toxicity Assay. Rat bone marrow stem cells will be cultured in a 96-well-plate. On day 1, day 3, and day 7, the supernatant will be collected and used to determine lactate dehydrogenase (LDH) cytotoxicity according to the manufacturer's instructions (Pierce LDH Cytotoxicity Assay Kit, Thermo Fisher Scientific AG, Reinach BL, Switzerland). Using a microplate reader, absorbance will be measured at 490 nm with background subtraction at 680 nm.

Live/Dead Staining in 3D Ex Vivo Blood Clots and Confocal Microscopy. Rat bone marrow stem cells will be cultured within 3D blood clots. At 1, 3, and 7 days the blood clots will be removed from the culture media and cut sagittally into halves before being immersed in serum-free medium containing 10 μM Calcein AM stock and 1 μM ethidium homodimer-1 (Thermo Fisher Scientific, Inc.) in a 24-well-plate. After an incubation of 3 hours at 4° C. and 1 hour at 37° C., 5% CO₂, and 100% humidity, the samples will be imaged up to a depth of 200 μm using a confocal microscope (Gantenbein-Ritter B, Sprecher C M, Chan S, Illien-Junger S, Grad S. Confocal imaging protocols for live/dead staining in three-dimensional carriers. Methods Mol Biol. 2011; 740: 127-40).

3D Cell Differentiation Assays. Cell-seeded blood clots will be cultured in either adipogenic, chondrogenic, or osteogenic medium (StemPro® Differentiation Kits, Thermo Fisher Scientific, Inc.) according to the manufacturer's protocol. Cell differentiation will be evaluated using qRT-PCR and customized TaqMan® PCR array plates (Thermo Fisher Scientific, Inc.).

Real Time Quantitative PCR (qRT-PCR). Ex vivo blood clots will be collected into microcentrifuge tubes, immediately snap-frozen in liquid nitrogen and stored at −80° C. RNA extractions will be performed using the QIAGEN RNeasy Plus Universal Tissue Mini (QIAGEN, Inc., Germantown, MD, USA) following the manufacturer's protocol. The concentration and quality of the RNA will be determined in a nanodrop spectrophotometer (ND-1000, Thermo Fisher Scientific, Inc.) and RNA integrity will be assessed using an Agilent 2100 Bioanalyzer (Agilent Technologies, Inc., Santa Clara, CA, USA) according to the manufacturer's protocol. The TaqMan™ High-Capacity RNA-to-cDNA Kit (Thermo Fisher Scientific, Inc.) will be used to reverse-transcribe 1 μg of extracted RNA into cDNA before customized TaqMan® PCR array plates (Thermo Fisher Scientific, Inc.) will be used to analyze the expression of inflammation-, angiogenesis- and osteogenesis-related genes.

As disclosed herein, the ability of bone marrow MSCs to survive in the presence of ecarin will be further investigated, as well as when seeded within ecarin-induced blood clots, to assess the biocompatibility of these scaffolds. Using various concentrations of coagulating enzyme derived from snake venom was not toxic to the MSCs added to an ex vivo blood clot. The enzyme, ecarin is highly purified. Thus, it can be expected that ecarin will not be toxic to the migrating cells once the ex vivo hematoma is implanted in vivo. Furthermore, in vivo experiments in a rat model were performed, which demonstrated that ecarin at 2 different concentrations (0.3 and 0.6 U/ml) did not cause toxicity. However, if ecarin prove to be toxic to the cells due to higher concentrations being required to create a blood clot with specific structural and biological properties for human subjects, calcium chloride or other coagulating factors described herein will be considered as clotting agents. As mentioned herein, thrombin has previously been used to activate the coagulation cascade in platelet rich plasma; however, this product showed poor performance in bone repair (Diesen D L, Lawson J H. Vascular. 2008; and Sands J J, Nudo S A, Ashford R G, Moore K D, Ortel T L. Am J Kidney Dis. 2000; 35: 796-801). This could be related to the fact that added thrombin to PRP was purely used as an activating agent, with the structural properties of the clot not being considered. In some aspects, a small dose of growth factors such as VEGF, PDGF, hFGF-2 or BMP-2 (other BMPs) can be added.

Scanning electron microscopy images depict the structural properties of ex vivo snake venom-induced ex vivo hematomas. These results show that cell viability was not significantly affected by the presence of the snake venom enzyme, suggesting biocompatibility.

### Example 9: To Investigate if an Ex Vivo Produced Ex Vivo Hematoma Inserted into a Large Bone Defect can Enhance the Regeneration Process of Bone in a Rat Femoral 5 Mm Large Critical Size Defect Model

Femoral defects of 5 mm created in groups of male SAS Fischer rats (10-12 weeks old, n=8-16; a pilot study n=4; ex vivo hematoma, BMP-2, PRP), and stabilized with external fixators as described herein. The ex vivo hematoma was implanted in the fracture gap to determine its ability to enhance the healing of large segmental bone defects. Two control groups were used to compare the bone healing process with the experimental group. Healing in the first control group was enhanced using recombinant human BMP-2 delivered on an absorbable collagen sponge, the same product that is currently used clinically (Infuse®, Medtronic plc., Minneapolis, MN, USA). This is a well-established research model using BMP-2, that has been reported to heal within 8 weeks (Glatt V, Bartnikowski N, Quirk N, Schuetz M, Evans C. Reverse Dynamization: Influence of Fixator Stiffness on the Mode and Efficiency of Large-Bone-Defect Healing at Different Doses of rhBMP-2. J Bone Joint Surg Am. 2016; 98: 677-87; Yasko A W, Lane J M, Fellinger E J, Rosen V, Wozney J M, Wang E A. The healing of segmental bone defects, induced by recombinant human bone morphogenetic protein (rhBMP-2). A radiographic, histological, and biomechanical study in rats. J Bone Joint Surg Am. The American Orthopedic Association; 1992; 74: 659-70; and Gantenbein-Ritter B, Sprecher C M, Chan S, Illien-Junger S, Grad S. Confocal imaging protocols for live/dead staining in three-dimensional carriers. Methods Mol Biol. 2011; 740: 127-40), and, therefore, a reduced number of animals was used (n=4). In the second control group, PRP was used to determine if the ex vivo hematoma has superior healing outcomes compared to a fibrin clot that is enriched with a high concentration of platelets. In the experimental group, the ex vivo hematoma was used to determine if the created scaffolds have the ability to regenerate bone defects. The merits of the ex vivo hematoma are the following: (1) osteoinductivity—important growth factors will persist for an extended period to stimulate new bone formation; and (2) osteoconductivity—a well-organized fibrin structure will create a microenvironment favorable for the migration of MSCs and early mineralization. It is anticipated that the successful completion of this proof-of-concept series of experiments will lead to the development of ex vivo hematoma that can act as a natural growth factor reservoir, as well as a biocompatible autologous scaffold, which will improve the healing of large segmental bone defects without the addition of growth factors, such as rhBMP-2. To evaluate this, the animals were monitored by weekly X-ray and euthanized at 8 weeks. After euthanasia, the healed defects were harvested for evaluation by micro-computed tomography (μCT; all samples) and will be used for histology/IHC (n=4/group; n=2 for BMP-2 group), and biomechanical testing (n=12/group; n=6 for BMP-2 group).

Surgery. Rat surgeries were performed as described herein.

Preparation of ex vivo hematoma for implantation. To create ex vivo blood clots, 5-10 mL whole blood was collected from anesthetized rats by cardiac puncture at the time of euthanasia. Immediately after collection the blood was mixed 9:1 with 4% sodium citrate solution to prevent coagulation. Ecarin was used at the previously determined concentrations of 0.3 and 0.6 U/ml to induce blood clotting in combination with 0.55 μg of rhBMP-2. The samples were allowed to clot for 45 min to 1 h at 22° C. (room temperature) before implantation into the 5 mm rat bone defects.

Platelet rich plasma (PRP) preparation and BMP-2. To prepare PRP from rat blood, 5-10 mL whole blood was collected from anesthetized rats by cardiac puncture at the time of euthanasia. Immediately after collection, the blood was mixed 9:1 with 4% sodium citrate solution to prevent coagulation. The whole blood was centrifuged at 150×g for 10 min at room temperature with a soft brake to separate the platelet layer from the plasma and red blood cells. The lower red blood cell layer was discarded while the middle platelet layer and the upper plasma layer was collected. The platelet number in whole blood and PRP was determined using a cell counting chamber to check the quality of the PRP. Calcium chloride was added to create a PRP gel, which was implanted into the defect gap. Recombinant human BMP-2 (5.5 μg) was applied to an absorbable collagen sponge carrier (Infuse™ Bone Graft, Medtronic, Minneapolis, MN, USA) in the shape of the bone defect, which was used as an implant.

Assessment of bone healing. Femora in each group of sixteen animals will be assessed in vivo by weekly X-ray and, after euthanasia, by μCT. Twelve samples will be subjected to biomechanical testing and four will be used for histology. For a pilot study, 4 animals per group were used.

X-ray. Bone healing was monitored by weekly radiography. Under general anesthesia as described in the surgical procedure, the rats were placed in a ventral position and the hind limbs laterally rotated to permit reproducible and standard images normal to the defect.

Micro-Computed Tomography (μCT). Femora will be scanned using a desktop micro-tomographic imaging system (Bruker Skyscan 1172, Belgium) equipped with a 10 mm focal spot microfocus X-ray tube. Femora will be scanned using a 16 μm isotropic voxel size at 75 keV energy and 250 ms integration time, with approximately 600 μCT slices per specimen. Evaluation will be applied in the 4 mm central defect region to ensure that no pre-existing cortical bone is included in the analyses. To evaluate the region of interest, the variables of the total cross-sectional volume of the defect (TV, mm³) and the bone volume (BV, mm³), bone volume fraction (BV/TV, %), bone mineral density (BMD, mg HA/ccm), and polar moment of inertia (pMOI, mm⁴) will be assessed. Images will be thresholded using an adaptive-iterative algorithm, and morphometric variables will be computed from the binarized images using direct 3D techniques that do not rely on any prior assumptions about the underlying structure.

Mechanical Testing. Following the non-invasive imaging, 12 specimens from each group will be tested in torsion to failure. The ends of each specimen will be embedded in polymethymethacrylate to provide an appropriate and reproducible gripping interface with the testing module. Specimens will be tested to failure under regular deformation control and at a constant deformation rate of 5 rad/min. Angular deformation and applied load data will be acquired at 10 Hz. The torque and rotation data will be used to compute the torsional stiffness and strength of the healed defects.

Histology of bone samples. Femora (n=4) will be fixed in ice-cold 4% paraformaldehyde for 48 hours, followed by decalcification in 14% EDTA for up to four weeks. Pins will be removed from the bones before embedding and sectioning. Fixed and decalcified tissues will be dehydrated in graded ethanol up to 100%, transferred to xylene, and embedded in paraffin. Five-micron paraffin sections will be placed on poly L-lysine coated slides, dried overnight and stained immediately, or stored at 4° C. Alternate sections will be stained with hematoxylin and eosin or safranin O and fast green before being examined by light microscopy. Safranin O is included to stain cartilage as part of monitoring the endochondral ossification process.

Power analysis and statistics. Sample sizes for all individual groups are based on coefficients of variation of 15% in the types of data collected and using an alpha level of 0.05 and power of 80% (beta=0.20). Power analysis revealed that n=8-16 animals per group will allow detection of significant differences between groups for each outcome parameter, based upon an effect size of 1.3 using the Student t-test. Previous experience with these rat models confirms that an n of 10 gives ample statistical power. The in vitro experiments will be performed in triplicates and compared for statistical significance using ANOVA tests. Sample size and power calculations were determined using version 4.0 of the nQuery Advice software program (Statistical Solutions, Boston, MA). Statistical analysis will be performed using SAS version 6.12 software (SAS Institute, Cary, NC). A two-tailed p<0.05 will be considered statistically significant.

Experiments to determine in vitro dose response are important to create an ex vivo hematoma with specific structural properties that mimic the structural properties of a naturally healing fracture hematoma. If healing cannot be achieved using the ex vivo blood clot alone, the ex vivo hematoma will be combined with either rat bone marrow mesenchymal stem cells, a significantly reduced amount of rhBMP-2 compared to the supra-physiological doses currently used clinically, or other growth factors.

As disclosed herein, the in vivo results from the in vivo study at the end of 8 weeks and 4 weeks (group with 0.6 U/ml+0.55 μg of BMP-2) bone defect healing times clearly demonstrate that whole blood+ecarin (0.1 U/mL) and Platelet Rich Plasma (PRP)+CalCl₂ (10 mM) do not enhance bone healing/regeneration at the concentrations of the coagulating factors tested. By contrast, when 0.3 U/mL of ecarin and either 1.1 μg or 0.55 μg of BMP-2 was added to the defects, the 5 mm femoral rat defects healed. It is important to note that BMP-2 delivered on the collagen sponge sold by Medtronic (Infuse™) using the same amounts of BMP-2 did not initiate the healing of bone defects. Interestingly, when using 0.6 U/mL ecarin+0.55 μg BMP-2, the healing was much better compared to that observed with the lower concentration of ecarin. This result seems to indicate that the ultrastructural properties of the blood clots (ex vivo hematomas) have a great influence on enhancing the healing of bone defects.

### Example 10: Biomimetic Hematoma: Influence of Coagulant and rhBMP-2 Concentration on Bone Healing Versus rhBMP-2/ACS

A range of concentrations of the coagulants, ecarin and calcium/thrombin, as well as BMP-2 were tested, and demonstrate the ability to initiate the healing of large segmental bone defects. The concentrations of ecarin tested included 0.3, 0.6, and 0.75 U/mL, and these concentrations successfully initiated healing; however, the concentration of 0.6 U/mL of ecarin showed the best result of the doses tested. Likewise, a combination of coagulants, 10 mM CaCl₂ and 0.5 U/mL thrombin, also effectively healed large bone defects in a manner similar to 0.6 U/mL of ecarin with 0.33 μg of BMP-2. The concentration of BMP-2 that consistently initiated healing of 5 mm bone defects in this rat model is 0.33 μg. This dose is 33 times lower than the standard dose of 11 μg, and this dose is 17 times lower than the lowest effective dose of BMP-2/ACS (ACS=absorbable collagen sponge) that efficiently heals large segmental bone in a 5 mm femoral defect rat model (5.5 μg). The other two doses tested that initiated the healing of bone defects were 0.165 and 0.0825 μg; however, the response with these doses was less consistent, and 75% and 50% respectively compared to 0.33 μg.

What is claimed is:

1. A composition comprising:
1) an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride; and
2) a bone substitute.

2. A composition comprising:
1) an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride; and
2) a bone substitute.

3. The composition of claim 1 or 2, wherein the ex vivo hematoma comprises fibrin fibers having a thickness of at least 150-300 nm±10%.

4. The composition of claim 1 or 2, wherein the bone substitute is demineralized bone matrix.

5. The composition of claim 1 or 2, wherein the bone substitute is derived from a biological product, a synthetic bone substitute or a combination thereof.

6. The composition of claim 5, wherein the biological product is a demineralized bone matrix, hydroxyapatite, or a coral.

7. The composition of claim 5, wherein the synthetic bone substitute is calcium sulfate, a calcium phosphate cement, β-tri-calcium phosphate ceramics, bioactive glasses, or a polymer.

8. The composition of claim 2, wherein the ex vivo hematoma further comprises sodium citrate.

9. The composition of claim 1 or 2, wherein the ex vivo hematoma further comprises an antibiotic.

10. The composition of any of the preceding claims, wherein the ex vivo hematoma further comprises one or more growth factors.

11. The composition of claim 10, wherein the one or more growth factors is bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof.

12. The composition of claim 1, wherein the whole blood comprises viable cells and one or more biological factors.

13. The composition of claim 12, wherein about 50% to 70% of the viable cells of the whole blood remain viable after formation of the hematoma.

14. The composition of any of the preceding claims, wherein the ex vivo hematoma further comprises a therapeutic agent.

15. The composition of claim 1, wherein the ex vivo hematoma comprises isolated whole blood, ecarin, and sodium citrate.

16. The composition of claim 1, wherein the ex vivo hematoma comprises isolated whole blood, calcium chloride, and sodium citrate.

17. The composition of claim 2, wherein the ex vivo hematoma comprises platelet rich plasma and ecarin.

18. The composition of claim 2, wherein the ex vivo hematoma comprises platelet rich plasma and calcium chloride.

19. The composition of claim 1, wherein the ex vivo hematoma comprises isolated whole blood, sodium citrate, and thrombin.

20. The composition of claim 1, wherein the ex vivo hematoma comprises isolated whole blood, calcium chloride, or oscutarin and calcium chloride, and sodium citrate.

21. The composition of any of the preceding claims, wherein the concentration of ecarin present in the ex vivo hematoma is at least 0.05 U/mL.

22. The composition of any of the preceding claims, wherein the concentration of ecarin present in the ex vivo hematoma is 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 U/mL.

23. The composition of any of the preceding claims, further comprising bone morphogenetic protein 2 (BMP-2).

24. The composition of claim 14, wherein the therapeutic agent is bone morphogenetic protein 2 (BMP-2).

25. The composition of claim 24, wherein the BMP-2 present in the ex vivo hematoma is at a dose of at least 0.01 mg.

26. The composition of claim 24, wherein the BMP-2 is a recombinant BMP-2.

27. The composition of claim 26, wherein the recombinant BMP-2 comprises human BMP-2.

28. The composition of any of the preceding claims, wherein the ex vivo hematoma further comprising growth factors, platelets, and cells.

29. The composition of any of the preceding claims, wherein the composition is formulated as a gel, a liquid, a powder, a paste, granules, or a putty.

30. The composition of any of the preceding claims, wherein the composition is formulated for local administration.

31. The composition of claim 23, wherein the amount of the ecarin present in the ex vivo hematomas is at least 0.05 U/mL; and the amount of BMP-2 present in the ex vivo hematoma is at least 0.01 mg.

32. The composition of any of the preceding claims, wherein the ratio of the ex vivo hematoma to bone substitute is from 1000:1 to 1:1000.

33. A device comprising the composition of any of the preceding claims.

34. A multi-compartment device comprising a first chamber comprising isolated whole blood and/or one or more growth factors, one or more bone substitutes, or a combination thereof; a second chamber comprising ecarin or calcium chloride; thrombin; or thrombin and calcium chloride.

35. A biomimetic scaffold comprising a scaffold and an ex vivo hematoma, wherein the ex vivo hematoma comprises: (a) isolated whole blood; (b) sodium citrate; and (c) ecarin; oscutarin and calcium chloride; calcium chloride; thrombin; or thrombin and calcium chloride.

36. A biomimetic scaffold comprising a scaffold and an ex vivo hematoma, wherein the ex vivo hematoma comprises: (a) platelet rich plasma, plasma, or plasma with red blood cells; and (b) ecarin; oscutarin and calcium chloride; calcium chloride; thrombin; or thrombin and calcium chloride.

37. The biomimetic scaffold of claim 35 or 36, wherein the ex vivo hematoma comprises fibrin fibers having a thickness of at least 150-300 nm±10%.

38. The biomimetic scaffold of claim 35 or 36, further comprising a bone substitute.

39. The biomimetic scaffold of claim 38, wherein the bone substitute is demineralized bone matrix.

40. The biomimetic scaffold of claim 39, wherein the bone substitute is derived from a biological product or a synthetic bone substitute.

41. The biomimetic scaffold of claim 40, wherein the biological product is a demineralized bone matrix, hydroxyapatite, or a coral.

42. The biomimetic scaffold of claim 40, wherein the synthetic bone substitute is calcium sulfate, a calcium phosphate cement, β-tri-calcium phosphate ceramics, bioactive glasses, or a polymer.

43. The biomimetic scaffold of any of claims 38-42, wherein the ratio of the ex vivo hematoma to bone substitute is from 1000:1 to 1:1000.

44. The biomimetic scaffold of any of the preceding claims, wherein the ex vivo hematoma further comprises sodium citrate.

45. The biomimetic scaffold of any of the preceding claims, wherein the ex vivo hematoma further comprises an antibiotic.

46. The biomimetic scaffold of any of the preceding claims, wherein the ex vivo hematoma further comprises one or more growth factors.

47. The biomimetic scaffold of any of the preceding claims, wherein the one or more growth factors is bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof.

48. The biomimetic scaffold of claim 35, wherein the isolated whole blood comprises viable cells and one or more biological factors.

49. The biomimetic scaffold of claim 48, wherein about 50% to 70% of the viable cells of the isolated whole blood remain viable after formation of the hematoma.

50. The biomimetic scaffold of any of the preceding claims, wherein the ex vivo hematoma further comprises a therapeutic agent.

51. The biomimetic scaffold of claim 35, wherein the ex vivo hematoma comprises isolated whole blood, ecarin and sodium citrate.

52. The biomimetic scaffold of claim 35, wherein the ex vivo hematoma comprises isolated whole blood, calcium chloride and sodium citrate.

53. The biomimetic scaffold of claim 36, wherein the ex vivo hematoma comprises platelet rich plasma and ecarin.

54. The biomimetic scaffold of claim 36, wherein the ex vivo hematoma comprises platelet rich plasma and calcium chloride.

55. The biomimetic scaffold of claim 35, wherein the ex vivo hematoma comprises isolated whole blood, sodium citrate, and thrombin.

56. The biomimetic scaffold of claim 35, wherein the ex vivo hematoma comprises isolated whole blood, calcium chloride, or oscutarin and calcium chloride, and sodium citrate.

57. The biomimetic scaffold of any of the preceding claims, wherein the concentration of ecarin present in the ex vivo hematoma is at least 0.05 U/mL.

58. The biomimetic scaffold of any of the preceding claims, wherein the concentration of ecarin present in the ex vivo hematoma is 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 U/mL.

59. The biomimetic scaffold of any of the preceding claims, wherein the ex vivo hematoma further comprises bone morphogenetic protein 2 (BMP-2).

60. The biomimetic scaffold of claim 50, wherein the therapeutic agent is bone morphogenetic protein 2 (BMP-2).

61. The biomimetic scaffold of claim 59, wherein the BMP-2 present in the ex vivo hematoma is at a dose of at least 0.01 mg.

62. The biomimetic scaffold of claim 59, wherein the BMP-2 is a recombinant BMP-2.

63. The biomimetic scaffold of claim 62, wherein the recombinant BMP-2 comprises human BMP-2.

64. The biomimetic scaffold of any of the preceding claims, wherein the ex vivo hematoma further comprises growth factors, platelets, and cells.

65. The biomimetic scaffold of any of the preceding claims, wherein the ex vivo hematoma is formulated as a gel, a liquid, paste, powder, a putty, or granules.

66. The biomimetic scaffold of any of the preceding claims, wherein the ex vivo hematoma is formulated for local administration.

67. The biomimetic scaffold of any of the preceding claims, wherein the amount of the ecarin present in the ex vivo hematomas is at least 0.05 U/mL; and the amount of BMP-2 present in the ex vivo hematoma is at least 0.01 mg.

68. The biomimetic scaffold of any of the preceding claims, wherein the scaffold is collagen, chitins, bioabsorbable polymers, nonabsorbable polymers such as PEEK, or titanium or a metallic alloy.

69. A method of promoting bone healing or producing bone replacement material or implants, the method comprising administering to a subject in need thereof a therapeutically effective amount of a composition of any of claims 1 to 32.

70. The method of claim 69, wherein the isolated whole blood comprises viable cells and one or more biological factors.

71. The method of claim 69, wherein the ex vivo hematoma comprises isolated whole blood, ecarin, and sodium citrate; isolated whole blood, calcium chloride, and sodium citrate; platelet rich plasma and ecarin; or platelet rich plasma and calcium chloride.

72. The method of claim 69, wherein the ex vivo hematoma further comprises bone morphogenetic protein 2 (BMP-2).

73. The method of claim 72, wherein the BMP-2 is a recombinant BMP-2.

74. The method of claim 73, wherein the recombinant BMP-2 comprises human BMP-2.

75. The method of claim 69, wherein the further comprises growth factors, platelets, and cells.

76. The method of claim 69, wherein the subject is a human.

77. The method of claim 69, wherein the composition is formulated as a clot or scaffold.

78. The method of claim 69, wherein the composition is formulated for local administration.

79. The method of claim 69, wherein the composition is administered locally, implanted, or delivered percutaneously.

80. The method of claim 69, wherein the composition is implanted.

81. The method of claim 71, wherein the amount of the ecarin present in the composition is at least 0.05 U/mL; and the amount of BMP-2 present in the composition is at least 0.01-5 mg.

82. The method of claim 69, wherein the subject has a skeletal defect.

83. The method of claim 82, wherein the skeletal defect is a small skeletal defect or a large segmental bone defect.

84. The method of claim 69, wherein the subject has one or more bone fractures.

85. The method of claim 69, wherein the subject has one or more bone injuries.

86. The method of claim 69, wherein the subject has a dental bone defect.

87. The method of claims 69-86, wherein the bone substitute is demineralized bone matrix.

88. The method of claim 69, wherein the bone substitute is derived from a biological product, a synthetic bone substitute or a combination thereof.

89. The method of claim 88, wherein the biological product is a demineralized bone matrix, hydroxyapatite, or a coral.

90. The method of claim 88, wherein the synthetic bone substitute is calcium sulfate, a calcium phosphate cement, β-tri-calcium phosphate ceramics, bioactive glasses, or a polymer.

91. The method of claim 69, wherein the ex vivo hematoma and the bone substitute are delivered percutaneously individually or in combination.

92. A method of constructing an implant, the method comprising:
a) dimensioning a depot implant in at least one of a shape and a size that facilitates implantation of the depot implant into a bone defect; and
b) structuring the depot implant to have a scaffold by introducing:
(i) isolated whole blood and sodium citrate; or platelet rich plasma, plasma, or plasma with red blood cells;
(ii) ecarin; oscutarin and calcium chloride; calcium chloride; thrombin; or thrombin and calcium chloride; and
(iii) a bone substitute to create the scaffold.

93. The method of claim 92, wherein the scaffold has a porosity of 55 to 75%.

94. The method of claim 92, wherein the scaffold comprises fibrin fibers having a thickness of at least 150-300 nm±10%.

95. The method of claim 92, wherein the shape of the depot implant is that of a cylinder or a sphere.

96. The method of claim 92, wherein the scaffold is constructed as a clot.

97. The method of claim 92, further comprising one or more growth factors.

98. The method of claim 92, wherein the one or more growth factors is bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof.

99. The method of claim 92, wherein the BMP-2 is introduced into the scaffold.

100. The method of claim 92, wherein the amount of ecarin present in the scaffold is at least 0.05 U/mL; and the amount of BMP-2 present in the scaffold is at least 0.01 mg.

101. The method of claim 92, wherein the scaffold resembles the size and shape of a given bone defect.

102. The method of claim 92, wherein the scaffold is chemotactic.

103. The method of claim 92, wherein the scaffold comprises viable blood cells and appropriate biological factors.

104. The method of claim 92, wherein the bone substitute is demineralized bone matrix.

105. The method of claim 92, wherein the bone substitute is derived from a biological product, a synthetic bone substitute or a combination thereof.

106. The method of claim 105, wherein the biological product is a demineralized bone matrix, hydroxyapatite, or a coral.

107. The method of claim 105, wherein the synthetic bone substitute is calcium sulfate, a calcium phosphate cement, β-tri-calcium phosphate ceramics, bioactive glasses, or a polymer.

108. A method of promoting bone healing in a subject, the method comprising implanting the biomimetic scaffold of any of claims 35-68 into a site of interest in the subject.

109. The method of claim 108, wherein the subject has a skeletal defect.

110. The method of claim 109, wherein the skeletal defect is a small skeletal defect or a large segmental bone defect.

111. The method of claim 108, wherein the subject has one or more bone fractures.

112. The method of claim 108, wherein the subject has one or more bone injuries.

113. The method of claim 108, wherein the subject has a dental bone defect.

114. A method of constructing a biomimetic scaffold, the method comprising:
a) dimensioning a scaffold in at least one of a shape and a size that facilitates implantation of the scaffold into a bone defect; and
b) combining the scaffold in a) with an ex vivo hematoma comprising:
(i) isolated whole blood and sodium citrate; or platelet rich plasma, plasma, or plasma with red blood cells; and
(ii) ecarin; oscutarin and calcium chloride; calcium chloride; thrombin; or thrombin and calcium chloride to create the biomimetic scaffold.

115. The method of claim 114, wherein the ex vivo hematoma has a porosity of 55 to 75%.

116. The method of claim 114, wherein the ex vivo hematoma comprises fibrin fibers having a thickness of at least 150-300 nm±10%.

117. The method of claim 114, wherein the shape of the scaffold is that of a cylinder or a sphere.

118. The method of claim 114, wherein the scaffold is collagen, chitins, bioabsorbable polymers, nonabsorbable polymers such as PEEK, or titanium or a metallic alloy.

119. The method of claim 114, wherein the ex vivo hematoma further comprises a bone substitute.

120. The method of claim 119, wherein the bone substitute is demineralized bone matrix.

121. The method of claim 119, wherein the bone substitute is derived from a biological product or a synthetic bone substitute.

122. The method of claim 120, wherein the biological product is a demineralized bone matrix, hydroxyapatite, or a coral.

123. The method of claim 121, wherein the synthetic bone substitute is calcium sulfate, a calcium phosphate cement, β-tri-calcium phosphate ceramics, bioactive glasses, or a polymer.

124. The method of claim 114, wherein the ex vivo hematoma further comprises one or more growth factors.

125. The method of claim 114, wherein the one or more growth factors is bone morphogenetic protein 2 (BMP-2), BMP-7, BMP-4, BMP-6, BMP-9, BMP-14, platelet-derived growth factor (PDGF), vascular endothelial growth factor (VEGF), fibroblast growth factor 2 (FGF-2), or a combination thereof.

126. The method of claim 114, wherein the BMP-2 is introduced into the ex vivo hematoma.

127. The method of claim 114, wherein the amount of ecarin present in the ex vivo hematoma is at least 0.05 U/mL; and the amount of BMP-2 present in the scaffold is at least 0.01 mg.

128. The method of claim 114, wherein the scaffold resembles the size and shape of a given bone defect.

129. The method of claim 114, wherein the scaffold is chemotactic.

130. The method of claim 114, wherein the ex vivo hematoma further comprises viable blood cells and appropriate biological factors.
